(19) United States
(12) Patent Application Publication
(10) Pub. No.: US 2018/0280637 A1
(43) Pub. Date: Oct. 4, 2018

(54) VAPORIZING DEVICE SYSTEM AND METHOD

(71) Applicant: ADVANCED GROW LABS TECHNOLOGIES, LLC, West Haven, CT (US)

(72) Inventors: Chris G. Mayle, Fairfield, CT (US); Stanley Scheufler, Encitas, CA (US); Mark Schwartz, Wauconda, IL (US); Brian Schwartz, Algonquin, IL (US)

(73) Assignee: ADVANCED GROW LABS TECHNOLOGIES, LLC, West Haven, CT (US)

(21) Appl. No.: 15/938,548

(22) Filed: Mar. 28, 2018

Related U.S. Application Data
(60) Provisional application No. 62/477,551, filed on Mar. 28, 2017.

(51) Int. Cl. A61M 15/00; A24F 47/00

(52) U.S. Cl. CPC A61M 15/0048; A61M 2205/3368; A61M 15/008; A24F 47/008

(57) ABSTRACT

Embodiments of the disclosure include an apparatus with a base. The apparatus also includes a cover arranged over the base, the cover comprising a plurality of compartments for storing an active pharmaceutical ingredient (API) within a chamber of each compartment of the plurality of compartments, each chamber being formed within the cover. Additionally, the apparatus includes a plurality of heaters, each respective heater of the plurality of heaters being positioned within the compartment for transmitting energy to the API. Also, the apparatus includes power contacts positioned on the base, the power contacts positioned to transmit received operational power to the plurality of heaters.

### CROSS-REFERENCE TO RELATED APPLICATIONS

This application claims priority to and the benefit of U.S. Provisional Application Ser. No. 62/477,551, filed Mar. 28, 2017, the full disclosure of which is hereby incorporated herein by reference in its entirety for all purposes.

### BACKGROUND

### Field of the Invention

The present invention relates to vaporizing devices. More particularly, the present invention relates to systems and methods to administer a metered dose via a vaporizing device.

### Description of Related Art

Vaporizing devices are utilized to heat an oil or extract (e.g., cannabis oil, tobacco oil, etc.) to generate an inhalable vapor for a user. Instead of igniting the cannabis and/or tobacco to facilitate transmission of the oils to the user, the vaporizing device heats the oils to a temperature that is below combustion, yet enables the active ingredients (e.g., tetrahydrocannabinol (THC), Cannabidiol (CBD), cannabinol (CBN), cannabavarin (THCV), cannabigerol (CBG), cannabichromene (CBC), delta-8-THC, cannabicyclol (CBL), cannabitriol (CBT), and cannabielsoin, etc.) to be converted into a vapor for inhalation and use by the user. Typically, vaporizing devices contain one or more heating elements positioned to transmit energy to the oils or extract to enable the user to receive a dosage of the active ingredients. However, it may be difficult to determine the dosage administered by the vaporizing devices due to the bulk storage of the oils or extract in a container. Further, dosage determinations based on inhalation times or volume may be challenging to monitor. As a result, inefficiencies arise with the dosing and treatment of a variety of ailments.

### SUMMARY

In an embodiment, an apparatus includes a base. The apparatus also includes a cover arranged over the base, the cover comprising a plurality of compartments for storing an active pharmaceutical ingredient (API) within a chamber of each compartment of the plurality of compartments, each chamber being formed within the cover. Additionally, the apparatus includes a plurality of heaters, each respective heater of the plurality of heaters being positioned within the compartment for transmitting energy to the API. Also, the apparatus includes power contacts positioned on the base, the power contacts positioned to transmit received operational power to the plurality of heaters.

In an embodiment, a system for vaporizing an active pharmaceutical ingredient (API) includes a body. The body includes a housing positioned within the body, the housing arranged to receive vaporized activated pharmaceutical ingredients (APIs) for inhalation. The body also includes a mouthpiece arranged at a first end of the body, opposite a second end, the mouthpiece being fluidly coupled to the housing to direct the vaporized API out of the housing. Additionally, the body includes a power supply to facilitate vaporization of the API. The system also includes a cartridge for storing the API, the cartridge is positioned within the housing and includes a base having a plurality of power contacts to electrically couple the cartridge to the power supply. The cartridge also includes a cover arranged over the base, the cover comprising a plurality of compartments for storing the API, each compartment having a chamber. Additionally, the cartridge includes a plurality of heaters, wherein a heater of the plurality of heaters is positioned within a compartment of the plurality of compartments for transmitting energy from the power supply to the API in the chamber.

In an embodiment, a method for using a vaporizing device includes receiving information from an identification arranged on a cartridge containing an active pharmaceutical ingredient (API). The method also includes activating a first compartment of a plurality of compartments arranged within the cartridge that house the API, wherein activating comprises converting the API into an inhalable vapor. The method further includes administering the dosage for inhalation by a user. The method also includes preparing a second compartment, different from the first compartment, for a second activation.

In another embodiment, a non-transitory computer-readable medium with computer-executable instructions stored thereon executed by one or more processors to perform a method to monitor dosages remaining in a cartridge includes receiving a first input, from at least one of a personal electronic device or a sever, indicative of a cartridge containing an active pharmaceutical ingredient (API) for use in treating one or more ailments. The method also includes determining a number of compartments arranged within the cartridge, each compartment including a metered dosage of API. The method further includes receiving a second input indicative of activating a compartment of the number of compartments, wherein activating comprises converting the API to an inhalable vapor. The method also includes tracking the compartment of the number of compartments that is activated, the activated compartment no longer containing the API after activation.

### BRIEF DESCRIPTION OF DRAWINGS

The file of this patent contains at least one drawing executed in color. Copies of this patent or patent application publication with color drawing(s) will be provided by the Office upon request and payment of the necessary fee.

The foregoing aspects, features, and advantages of the present invention will be further appreciated when considered with reference to the following description of embodiments and accompanying drawings. In describing the embodiments of the invention illustrated in the appended drawings, specific terminology will be used for the sake of clarity. However, the invention is not intended to be limited to the specific terms used, and it is to be understood that each specific term includes equivalents that operate in a similar manner to accomplish a similar purpose.

FIG. 1 is a block diagram of an embodiment of a vaporizing device, in accordance with the present disclosure;

FIG. 2 is a top plan view of an embodiment of a cartridge, in accordance with the present disclosure;

FIG. 3 is a bottom plan view of the cartridge of FIG. 2, in accordance with the present disclosure;

FIG. 4 is a cross-sectional perspective view of the cartridge of FIG. 2 taken along line 4-4, in accordance with the present disclosure;

FIG. 5 is an exploded top perspective view of the cartridge of FIG. 2, in accordance with the present disclosure;

FIG. 6 is an exploded bottom perspective view of the cartridge of FIG. 2, in accordance with the present disclosure;

FIG. 7 is a top perspective view of an embodiment of a cartridge, in accordance with the present disclosure;

FIG. 8 is a cross-sectional side perspective view of the cartridge of FIG. 7, in accordance with the present disclosure;

FIG. 9 is an exploded top perspective view of the cartridge of FIG. 7, in accordance with the present disclosure;

FIG. 10 is a top perspective view of an embodiment of a cartridge, in accordance with the present disclosure;

FIG. 11 is a cross-sectional side perspective view of the cartridge of FIG. 10, in accordance with the present disclosure;

FIG. 12 is an exploded top perspective view of the cartridge of FIG. 10, in accordance with the present disclosure;

FIG. 13 is a schematic diagram of the cartridge of FIG. 7, in accordance with the present disclosure;

FIG. 14 is a schematic diagram of the cartridge of FIG. 7, in accordance with the present disclosure;

FIG. 15 is a schematic diagram of the cartridge of FIG. 7, in accordance with the present disclosure;

FIG. 16 is a block diagram of an embodiment of a communication network, in accordance with the present disclosure;

FIG. 17 is a flow chart of an embodiment of a method of using a vaporizing device, in accordance with the present disclosure;

FIG. 18 is a graphic representation of a temperature over time, in accordance with the present disclosure;

FIG. 19 is a block diagram of an embodiment of hardware architecture for the vaporizing device of FIG. 1, in accordance with the present disclosure;

FIG. 20 is a flow chart of an embodiment of a method for activating a compartment of the vaporizing device of FIG. 1, in accordance with the present disclosure;

FIG. 21 is a schematic diagram of an embodiment of a user profile, in accordance with the present disclosure;

FIG. 22 is a schematic diagram of an embodiment of a server communicating with personal electronic devices, in accordance with the present disclosure; and

FIG. 23 is a schematic diagram of an embodiment of a dosage profile, in accordance with the present disclosure.

### DETAILED DESCRIPTION

The foregoing aspects, features, and advantages of the present invention will be further appreciated when considered with reference to the following description of embodiments and accompanying drawings. In describing the embodiments of the invention illustrated in the appended drawings, specific terminology will be used for the sake of clarity. However, the invention is not intended to be limited to the specific terms used, and it is to be understood that each specific term includes equivalents that operate in a similar manner to accomplish a similar purpose.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments,” or “other embodiments” of the present invention are not intended to be interpreted as excluding the existence of additional embodiments that also incorporate the recited features. Furthermore, reference to terms such as “above,” “below,” “upper”, “lower”, “side”, “front,” “back,” or other terms regarding orientation are made with reference to the illustrated embodiments and are not intended to be limiting or exclude other orientations.

Embodiments of the present disclosure include a vaporizing device having a cartridge including an active pharmaceutical ingredient (API). In certain embodiments, the cartridge is disposable or replaceable and includes individual compartments storing a metered amount of API, for example, via a wick for later vaporization and inhalation by a user. Furthermore, in certain embodiments, each compartment includes an individual heater to transmit heat energy to the API to initiate vaporization. Accordingly, metered amounts of API may be selectively activated and vaporized, thereby enabling users to accurately dose the API. In certain embodiments, the vaporized API exits the compartment (e.g., a chamber of the compartment) and fills a housing forming at least a portion of a void space to receive the vapor. However, in certain embodiments, multiple components of the vaporizing device may be utilized to form the form void space. Thereafter, the user can selectively administer the vapor. For example, the vaporizing device may include a mouthpiece and a flow restrictor that maintains the vapor within the housing until the user is ready for administration. Moreover, the user can select how much of the vapor to inhale at a given time due to the flow restrictor. In this manner, the user may dose a metered amount of API at a comfortable rate.

In certain embodiments, the oil mixture includes cannabis oil having a quantity of active tetrahydrocannabinol (THC) and/or cannabidiol (CBD) or any other active ingredient. The oil mixture may also include an excipient, such as polyethylene glycol (PEG), added in a quantity proportional to the quantity of active THC and/or CBD in the oil such that a cartridge containing a cannabis oil having a higher concentration of THC and/or CBD contains a greater amount of PEG than a cartridge containing a cannabis oil having a lower concentration of THC and/or CBD. It should be appreciated that the active THC and/or CBD can be formulated in a variety of configurations to provide relief to one or more ailments suffered by a user. For example, the oil mixture may have a 1:1 ratio of THC to CBD, a 1:2 ratio, a 1:5 ratio, a 1:50 ratio, a 2:1 ratio, a 5:1 ratio, a 50:1 ratio, or any other reasonable ratio of THC to CBD. As such, the oil mixture can be formulated to enhance the medicinal properties based on the response of one or more ailments.

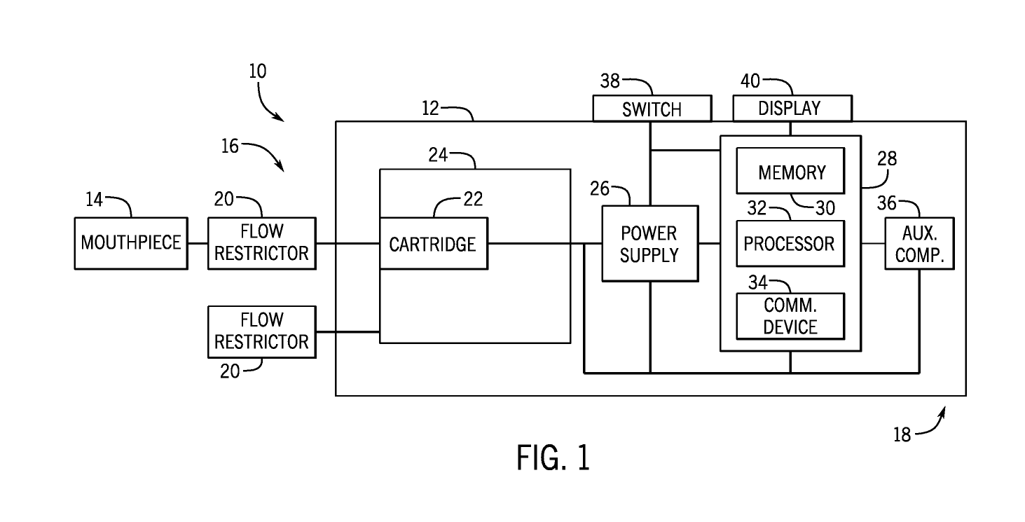

FIG. 1 is a schematic block diagram of an embodiment of a vaporizing device 10 for administering an amount (e.g., a dose, a targeted dose, a therapeutic dose, a metered dose, etc.) of an active pharmaceutical ingredient (API), such as an oil or extract (e.g., an oil mixture, cannabis oil, tobacco oil, other inhalable medicines, etc.). However, it should be noted that, in certain embodiments, the API may not be oil. For example, the API may include a powdered inhalable substance (e.g., an anti-inflammatory or steroid), or a mist (e.g., an inhalable vaccine, an anti-inflammatory, a steroid, etc.). In the illustrated embodiment, the vaporizing device 10 includes a body 12 with a mouthpiece 14 fluidly coupled to the body 12 at a first end 16, opposite a second end 18. For example, the mouthpiece 14 may include an opening which enables fluid communication with the body 12 such that a vapor generated by the vaporizing device 10 may be received by a user via the mouthpiece 14.

In the illustrated embodiment, a flow restrictor 20 is arranged between the mouthpiece 14 and the body 12. In certain embodiments, the flow restrictor 20 functions to block or restrict flow between the mouthpiece 14 and the body 12. For example, the flow restrictor 20 may include a one-way flow valve, such as a ball check valve (e.g., a spring-loaded ball check), a diaphragm check valve, a swing check valve, a stop-check valve, a lift-check valve, an in-line check valve, a duckbill valve, or the like. In the illustrated embodiment, the flow restrictor 20 restricts flow from the body 12 to the mouthpiece 14. For example, in embodiments where the flow restrictor 20 is a ball check valve, a ball arranged within the valve be driven toward an opening via a spring. Pressure from the mouthpiece 14 (e.g., suction pressure) may collapse the spring, thereby moving the ball and enabling flow from the body 12 to the mouthpiece 14. In this manner, flow may be restricted between the mouthpiece 14 and the body 12. Furthermore, in certain embodiments, the flow restrictor 20 may incorporate one or more sensors to facilitate functionality of the flow restrictor 20. For example, the flow restrictor 20 may include a pressure sensor that opens the valve when suction pressure at the mouthpiece 14 is detected. Accordingly, it should be appreciated that a variety of methods may be utilized to restrict flow between the mouthpiece 14 and the body 12.

In the illustrated embodiment, the body 12 houses a variety of components that facilitate operation of the vaporizing device 10. For example, in the illustrated embodiment, a cartridge 22 is arranged within a housing 24, both of which are positioned within the body 12. As will be described below, the housing 24 at least partially forms an annulus or void space around the cartridge 22 that enables vapor generated via heating of the API within the cartridge 22 to fill the housing 24 for later inhalation by a user. Furthermore, in certain embodiments, the cartridge 22 may interact with the housing 24 to form at least a portion of the void space. Additionally, in certain embodiments, the body 12 may not encompass both the cartridge 22 and the housing 24. For example, portions of the cartridge 22 and/or housing 24 may not be within the body 12. In the illustrated embodiment, the body 12 also includes a power supply 26 (e.g., a battery), a controller 28 with a memory 30, a processor 32, and a communication device 34, and an auxiliary component 36. Furthermore, in the illustrated embodiment, the body 12 has a switch 38 and display 40. As will be described below, the components of the vaporizing device 10 may be utilized to provide a measured dose of the API to a user.

In the illustrated embodiment, the cartridge 22 is positioned within the housing 24 such that the void space is arranged about the cartridge 22. It should be appreciated that that the void space need not fully encompass the cartridge 22. For example, the void space may be arranged over a single side of the cartridge, over multiple (but not all sides), or in any other reasonable orientation. In the illustrated embodiment, the cartridge 22 is communicatively coupled to the power supply 26. It should be appreciated that other components to facilitate the transfer of electrical energy from the power supply 26 to the cartridge, such as converters and the like, may be included. As such, the power supply 26 supplies electrical energy to the cartridge 22 to enable the vaporization of the API for subsequent inhalation by the user. Moreover, the cartridge 22 is communicatively coupled to the controller 28 and the auxiliary component 36. As will be described below, the controller 28 may be utilized to adjust or monitor the vaporization temperature of the API and to track remaining or implemented dosages from the cartridge 22. Furthermore, in the illustrated embodiment, a second flow restrictor 20 is coupled to the housing 24 to regulate the flow of air into the housing 24. As will be described in detail below, as compartments of the cartridge 22 fill with vapor, the second flow restrictor 20 may be utilized to equalize the pressure in the housing 24 as the user draws the vapor out through the mouthpiece 14.

In certain embodiments, the cartridge 22 is removable from the body 12. For example, the body 12 may include one or more openings to receive and secure the cartridge 22. As such, the cartridge 22 may be removed and discarded after its useful life has ended, for example, once the API is fully used. Moreover, embodiments with removable cartridges 20 enable the user to utilize multiple cartridges 20 having different APIs. For example, in the case where the API is cannabis oil, the user may utilize different types of cannabis oil at different times of the day, or to treat multiple ailments.

In the illustrated embodiment, the power supply 26 is arranged proximate the cartridge 22 to enable electrical communication between the power supply 26 and the cartridge 22. For example, the power supply 26 provides electrical energy to the cartridge 22 to enable vaporization of the API for inhalation by the user. In certain embodiments, the power supply 26 is a primary cell battery (e.g., zinc-carbon, alkaline, etc.), a rechargeable battery (e.g., nickel-cadmium, nickel-zinc, nickel metal hydride, lithium-ion, etc.), or any other type of suitable power source, such as a generator operable by the suction pressure from the mouthpiece 14. As illustrated, the switch 38 is communicatively coupled to the power supply 26. In certain embodiments, activation of the switch 38 (e.g., pressure on a tactile switch, sliding a contact, etc.) transmits an electrical signal to the power supply 26 to transmit electrical energy to the cartridge 22 for vaporization of the API. However, in certain embodiments, the switch 38 transmits a signal to the controller 28, which as a result of the signal, instructs the power supply 26 to transmit the electrical energy to the cartridge 22.

In the illustrated embodiment, the controller 28 is communicatively coupled to the cartridge 22, the power supply 26, the auxiliary component 36, the switch 38, and the display 40. Furthermore, as shown, the controller 28 includes the memory 30. In certain embodiments, the memory 30 is a computer-readable medium (e.g., a machine readable medium, a propagated-signal medium, and/or a signal-bearing medium) that may comprise any device that includes, stores, communicates, propagates, or transports software for use by or in connection with an instruction executable system, apparatus, or device. The machine-readable medium may selectively be, but not limited to, an electronic, magnetic, optical, electromagnetic, infrared, or semiconductor system, apparatus, device, or propagation medium. A non-exhaustive list of examples of a machine-readable medium would include an electrical connection (e.g., having one or more wires), a portable magnetic or optical disk, a volatile memory such as a Random Access Memory (RAM), a Read-Only Memory (ROM), an Erasable Programmable Read-Only Memory (EPROM or EEPROM, a non-volatile memory such as flash memory (e.g., NAND or NOR-type flash memory) or solid-state drives, or an optical fiber. A machine-readable medium may also include a tangible medium upon which software is printed, as the software may be electronically stored as an image or in another format (e.g., through an optical scan), then compiled, and/or interpreted or otherwise processed. The processed medium may then be stored in a computer and/or machine memory. Moreover, the processor 32 may include one or more micro-processors that perform the machine-readable instructions printed on the memory 3.

In certain embodiments, the controller 28 is utilized to monitor the use of the cartridge 22. That is, the controller 28 may count or record the number of times the cartridge 22 is used, thereby enabling the controller 28 to monitor the amount of API remaining within the cartridge 22 to facilitate notifications to the user, for example, via the display 40, of the remaining API. Furthermore, in the illustrated embodiment, the controller 28 includes the communication device 34, for example a Wi-Fi transceiver, a BLUETOOTH transceiver, a near field communication (NFC) transceiver, or a combination thereof. As will be described below, the communication device 34 enables communication between the vaporizing device 10 and one or more personal electronic devices, servers, computers, or the like.

In the illustrated embodiment, the controller 28 is communicatively coupled to the switch 38 and the display 40. As such, in certain embodiments, activation of the switch 38 may send an electrical signal to the controller 28 to transmit instructions to one or more components of the vaporizing device 10. For example, the controller 28 may instruct the power supply 26 to send electrical energy to the cartridge 22 or to transmit dosing information to the display 40 for the user to see. Furthermore, in certain embodiments, the controller 28 may also transmit or receive instructions from the auxiliary component 36. For example, in embodiments where the auxiliary component 36 is a vibrator, the controller 28 may instruct the auxiliary component 36 to vibrate to indicate that the vaporizing device 10 is ready for use by the user. Further, in other embodiments, the auxiliary component 36 may be one or more components such as sensors, timers, or the like that can send and receive information to and from the controller 28. Additionally, the auxiliary component 36 may be a temperature controller that controls the temperature at which the API is vaporized. In this manner, operation of the vaporizing device 10 may be monitored and controlled.

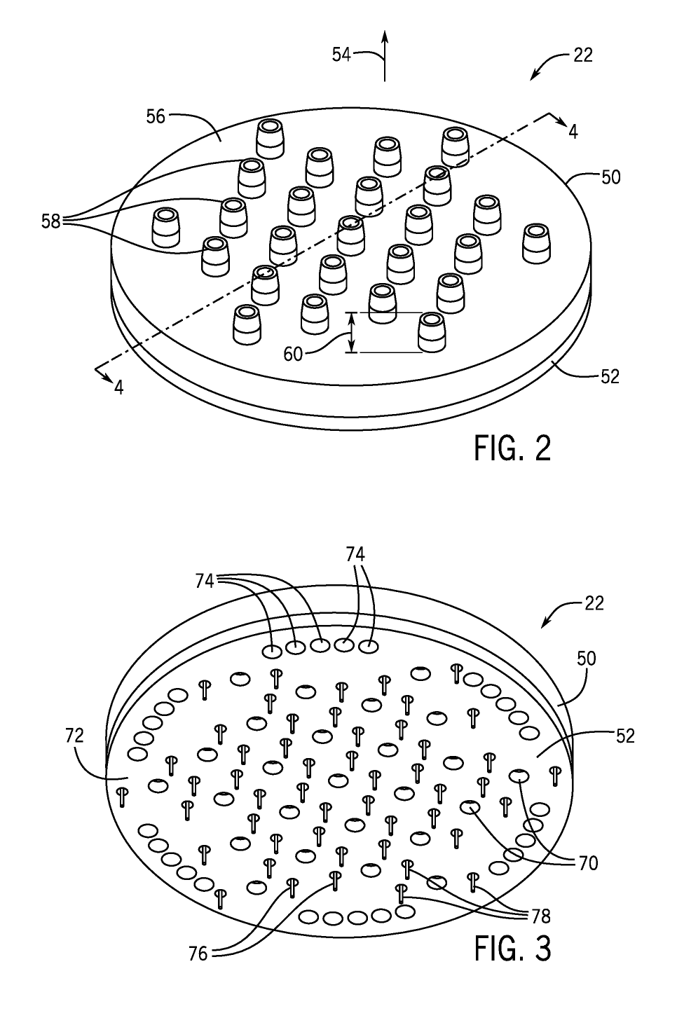

FIG. 2 is a top perspective view of an embodiment of the cartridge 22. As described above, the cartridge 22 may be utilized to store a metered dosage (e.g., predetermined dosage, individual dosage, measured dosage, etc.) of the API. As used herein, metered dosage refers to a preselected quantity of API. Furthermore, in embodiments, the cartridge 22 is disposable and/or replaceable. That is, once the stored API is full used then the cartridge 22 may be discarded by the user and replaced with a new cartridge 22 containing the API. As such, the user will not be refilling or modifying the cartridges and, instead, will receive the metered dosage with each use. In the illustrated embodiment, the cartridge 22 includes a cover 50 and a base 52. As shown, the cover 50 and base 52 each have a generally circular circumference and are axially aligned along a longitudinal axis 54. However, it should be appreciated that, in certain embodiments, the cartridge 22 will not be generally circular. For example, the cartridge 22 may be rectangular, ovular, arcuate, triangular, egg-shaped, cylindrical, or any other reasonable shape. Moreover, in certain embodiments, the cover 50 may not be included. In certain embodiments, the cover 50 is formed from a material capable of withstanding high temperatures, such as a high temperature or engineered plastics like Vespel, Torlon, Ryton, Nylon 6, polyamids, polycarbonates, polyetheretherketone, polyethylene terephthalate, polytetrafluoroethylene, or the like. As will be described below, the temperature insulating properties of the plastics facilitate forming individually vaporized compartments of API within the cover 50. The cover 50 is mated with the base 52, which may be formed from a silicon wafer or other semi-conductor to include one or more power contacts to enable coupling and communication with the controller 28, for example, via pogo pins or the like.

In the illustrated embodiment, the cover 50 includes a surface 56 having a plurality of outlets 58 extending axially away from the surface 56. As will be described below, vapor from the API may flow through the outlets 58 and into the housing 24 before inhalation by the user. In the illustrated embodiment, the outlets 58 are generally cylindrical and have an axial outlet height 60. However, it should be appreciated that, in certain embodiments, the axial outlet height 60 may be omitted and the outlets 58 may be formed by holes extending through the cover 50. The illustrated embodiment includes 25 outlets 58, which correspond to 25 chambers housing API. However, it should be appreciated that other embodiments may have more or fewer outlets. For example, the cartridge 22 may include 5, 10, 15, 20, 30, 35, 40, 45, 50, or any suitable number of outlets 58. As will be described below, the cartridge 22 holds the API within one or more chambers for vaporization and eventual output through the respective outlets 58.

FIG. 3 is a bottom perspective view of the cartridge 22. As described above, the base 52 is joined to the cover 50. In the illustrated embodiment, the base 52 includes intakes 70 that extend through a bottom surface 72 of the base 52 toward a chamber that includes the API for vaporization. For example, in certain embodiments, the intakes 70 will receive air (e.g., from suction at the mouthpiece 14, from air outside of the body 12, etc.) to drive the vaporized API toward the mouthpiece 14 for inhalation by the user as the pressure in a void space surrounding the cartridge 22 is normalized. In the illustrated embodiment, there are 25 intakes 70, corresponding to the number of outlets 58. However, as described above, there may be more or fewer intakes 70. Moreover, in certain embodiments, the number of intakes 70 may be greater than or less than the number of outlets 58.

In the illustrated embodiment, power contacts 74 are arranged on the bottom surface 72 to enable coupling of the cartridge 22 to the controller 28. For example, as described above, the power contacts 74 may be utilized to send and/or receive signals between the controller 28 or to receive electrical energy from the power supply 26 for vaporizing the API. Furthermore, in the illustrated embodiment, the bottom surface 72 of the base 52 also includes passages 76 through which a heater 78 (e.g., chamber heater, compartment heater, etc.) extend. As will be described below, in certain embodiments, the heater 78 transmits the electrical energy received from the power supply 26 to the API positioned within the chamber of the cover 50, thereby enabling the vaporization and subsequent inhalation of the API. Furthermore, as will be described below, the heater 78 illustrated in FIG. 3 is one non-limiting example and that, in various embodiments, other types of heaters 78 may be utilized. For example, in various embodiments, the heater 78 may include a ceramic or otherwise thermally conductive plate or fitting that transmits thermal energy to an object that includes combustible or otherwise vaporizable material, such as the API. In the illustrated embodiment, there are twice as many passages 76 as there are intakes 70 because each of the ends of the heater 78 has a separate passage 76. However, in other embodiments, there may be more or fewer passages 76.

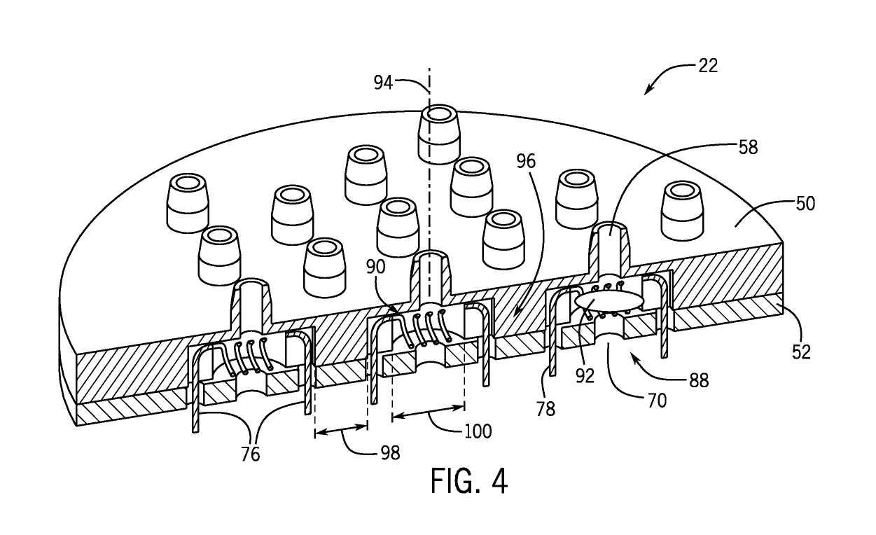

FIG. 4 is a partial perspective sectional view of the cartridge 22 taken along line 4-4. As described above, in the illustrated embodiment, the cartridge 22 includes the cover 50 and the base 52 to thereby form a compartment 88 to receive and store the API within a chamber 90 of the compartment 88. As used herein, the compartment 88 refers to the collective space formed by the cover 50 and/or the base 52 for receiving and storing the API and may, in certain embodiments, include the chamber 90, wick 92, heater 78, and associated passages. It should be appreciated that the API has been omitted from certain compartments 88 in FIG. 4 for clarity. However, in certain embodiments, the API may be arranged as part of a wick 92 (e.g., chamber wick, compartment wick) positioned within the chambers 90. As illustrated, the chamber 90 is an open area in the cover 50 holding the wick 92. However, it should be appreciated that, in certain embodiments, the chamber 90 may be formed in the base 52 and in both the cover 50 and the base 52. The wick 92 may be a porous, absorbent material, such as a fiber like cotton or hemp, that receives the energy from the power supply 26 via the heater 78 to vaporize the API. As a result, the API may be converted from an oil or abstract soaked onto the wick 92 into an inhalable substance for the user. It should be appreciated that, in certain embodiments, the API may be an oil that is viscous and therefore will not drip and/or separate from the wick 92. However, in embodiments where the API is not viscous, the intake 70 and/or outlet 58 may include a one-way valve, such as the valves described above with respect to the flow restrictor 20, to prevent leakage or drainage from the chamber 90. Additionally, in various embodiments, the wick 92 may not be utilized. For example, the wax, oil, or other material incorporating the API may be directly positioned within the compartment 88 to receive heat energy from the heater 78, which as described above may be any type of heater, such as a ceramic plate, that transmits thermal energy to the API.

In the illustrated embodiment, the compartments 88 are formed in the cover 50 and correspond to the outlets 58. That is, each outlet 58 is substantially aligned with the respective compartment 88 such that API vaporized within the chambers 90 will be fluidly coupled to a respective outlet 58 for inhalation by the user. For example, in the embodiment illustrated in FIG. 4, the outlet 58 and the intake 70 are coaxial along a compartment axis 94. As a result, as the energy from the power supply 26 is transmitted to the heater 78, and subsequently to the wick 92, the heat will vaporize the API to create an inhalable substance, such as a vapor, and be carried from the compartment 88 via the air flowing through the intake 70. However, it should be appreciated that in certain embodiments the expansion of the API caused by the conversion to the gaseous state may be sufficient to draw the inhalable substance or vapor from the compartment 88 for inhalation by the user.

As illustrated in FIG. 4, the heater 78 extends through the passages 76 and into the compartment 88 to coil (e.g., wind about) the wick 92 within the chamber 90. By winding around the wick 92, a larger surface area of the wick 92 is exposed to the heater 78, thereby facilitating improved heat conduction for vaporization. In other words, heat energy from the heater 78 is transferred to the wick 92 faster because more areas of the wick 92 are directly exposed to the heat from the heater 78. However, it should be appreciated that, in other embodiments, the heater 78 may be not in the form of a coiled wire. For example, the heater 78 may be a plate that extends along the compartment 88 to facilitate heat transfer to the wick 92. Moreover, in certain embodiments, the heater 78 may be in the form of a heating coil and blower to generate convective heat transfer throughout the compartment 88 to vaporize the API. Moreover, the compartment 88 may act at least partially as the heater 78 by being formed or coated in a thermally conductive material, such as ceramic. Additionally, a plate or other thermally conductive material or fitting may be arranged within the compartment 88 to transmit thermal energy to the API to vaporize the API. In this manner, the heater 78 is utilized to individually heat the wick 92 positioned within the compartment 88. In the illustrated embodiment, each compartment 88 includes an individual heater 78 to facilitate vaporization of the API within the chamber 90. However, it should be appreciated that, in certain embodiments, heaters 78 may be utilized to heat the API in one or more compartments 88. For example, each row of compartments 88 may utilize the same heater 78.

As described above, in certain embodiments, the cover 50 is manufactured from a heat-resistant material, such as an engineered plastic, to enable combustion and vaporization of API in one compartment 88 without heating and vaporizing the API in the adjacent compartments 88. For example, in the illustrated embodiment, a barrier 96 having a barrier width 98 is arranged between adjacent compartments 88. As a result, heat energy within the heaters 78 is not transferred to adjacent compartments 88. In certain embodiments, the barrier width 98 is less than a compartment width 100. However, in other embodiments, the barrier width 98 may be substantially equal to the compartment width 100 or greater than the compartment width 100. As illustrated, the barrier 96 may also be present in the base 52 to further block inadvertent heat transfer between adjacent compartments 88.

In operation, the wick 92 will be saturated with a metered amount (e.g., predetermined amount) of API. For example, each wick 92 may include 1.0 mg API, 1.5 mg API, 2.0 mg API, 2.5 mg API, 3.0 mg API, or any other reasonable amount of API. As such, when the wick 92 within the individual compartments 88 is vaporized, the metered amount of API will be released for inhalation by the user. Thereafter, the user may administer the dosage of API at a rate chosen by the user. For example, a user may not be able to inhale the entire amount of API produced from the compartment 88 in a single breath. Accordingly, the user may take multiple breaths, or pulls, of the vaporizing device 10 to receive the metered dose of API. Not only is dosing easier for the user, but the amount of API utilized by the patient is easier to track because each compartment 88 has a metered, measured, known dosage of API. Accordingly, treatment options, for example with medical marijuana, can be closely monitored and adjusted by compartment 88. That is, because the cartridge 22 illustrated in FIG. 4 includes individual and independent compartments 88 each having an individual chamber 90, administration of the API may be closely controlled through activation of the individual, independent compartments 88.

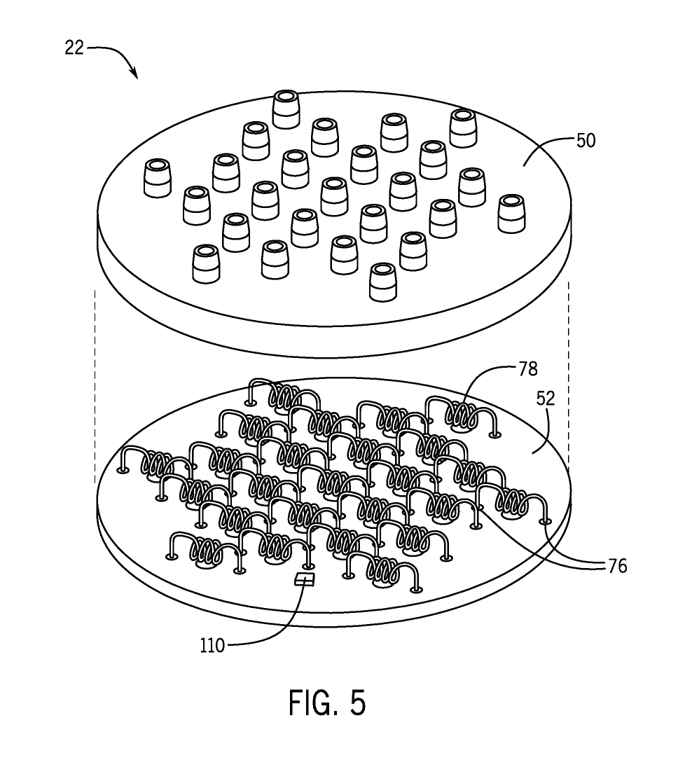

FIG. 5 is an exploded top perspective view of the cartridge 22. As described above, the cover 50 is arranged over the base 52 to form the cartridge 22. Moreover, the cover 50 includes the compartments 88 for receiving the heater 78 and the wick 92 (not pictured for clarity). In the illustrated embodiment, each compartment 88 includes a separate, individually controlled heater 78 to transfer heat to the wick 92 to vaporize the API for inhalation by the user. Moreover, as illustrated in FIG. 5, in certain embodiments the controller 28 includes identification 110. For example, in the illustrated embodiment, EEPROM memory is positioned on the base 52 for identification of the cartridge 22, by the vaporizing device 10 or, for example, a personal electronic device. In certain embodiments, the EEPROM stores information related to the cartridge 22, such as date the cartridge 22 was made, type of API, serial numbers, metered dosage per compartment 88, etc. However, it should be appreciated that, in other embodiments, different types of identifications 110 may be utilized. For example, as described above, identification of the cartridge may be communicated to the vaporizing device 10 via RFID or other near field communication to read information from the cartridge 22, thereby enabling communication between the vaporizing device 10 and the cartridge 22. For example, in certain embodiments, the vaporizing device 10, via the controller 28, may track the number of compartments 88 that have been used. As a result, the controller 28 may relay the information to the user via the display 40 or via a personal electronic device, and furthermore instruct different compartments 88 to be used or fired the next time the user administers a dose. In this manner, the quantity of API remaining can be tracked, which may be utilized to remind the user to purchase additional supplies when running low.

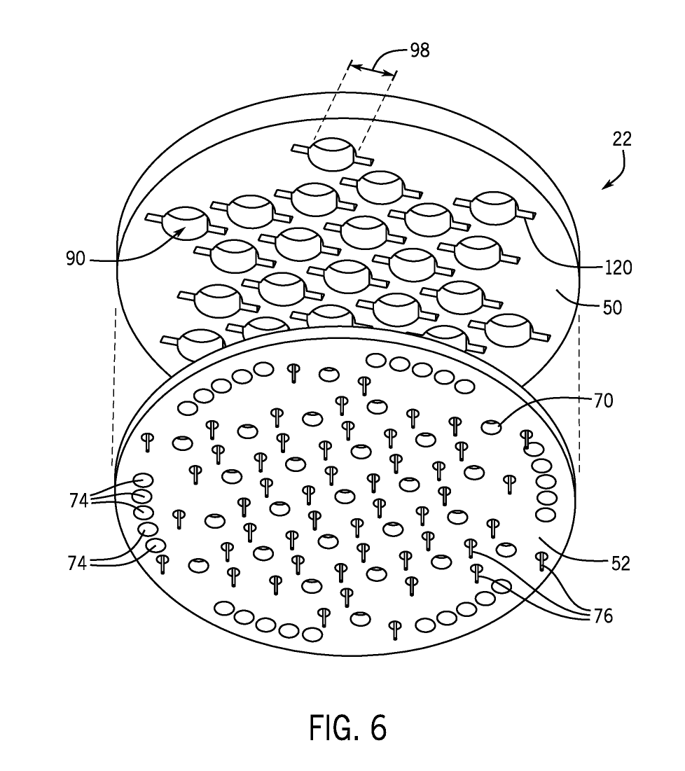

FIG. 6 is an exploded bottom perspective view of the cartridge 22. In the illustrated embodiment, the compartments 88 are formed in the cover 50 and have a generally circular shape with notches 120 for the heaters 78. However, it should be appreciated that, in certain embodiments, the compartments 88 may also be partially formed in the base 52 or fully formed in the base 52. Moreover, the notches 120 may not be generally rectangular, as illustrated, and may be round or any other reasonable shape. Furthermore, in certain embodiments, the notches 120 may be omitted and the heater 78 may be arranged directly within the compartment 88. In this manner, the cartridge 22 may be assembled for use.

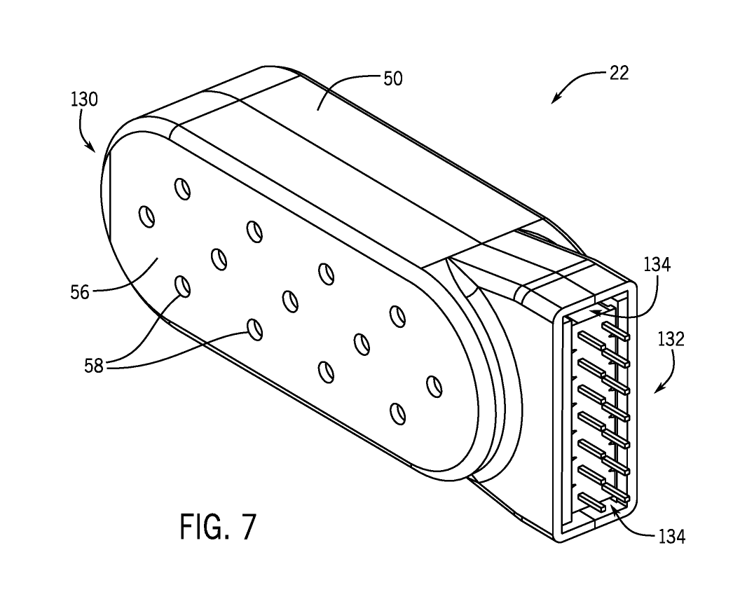

FIG. 7 is a perspective view of an embodiment of the cartridge 22. As shown, the cartridge 22 is elongated with a closed end 130 and a connective end 132. The connective end 132 includes one or more connectors, such as pogo pins or the like, to facilitate connection with the vaporizing device 10, namely to the controller 28. In the illustrated embodiment, the outlets 58 are arranged along the surface 56 of the cover 50 of the cartridge 22. In certain embodiments, the outlets 58 are also arranged along the back side of the cartridge 22 (not visible in FIG. 7), thereby forming a stacked cartridge which will be described in detail below. As described above, the cartridge 22 includes multiple compartments 88 housing an individual, measured dose of API. In certain embodiments, each compartment 88 includes the individual heater 78 to facilitate vaporization of the API. Furthermore, as described above, in certain embodiments the cartridge 22 may receive an air intake to facilitate transportation of the vaporized API out of the cartridge 22. For example, as shown in FIG. 7, air intakes 134 may be positioned proximate the contacts of the connective end 132. As a result, air may flow into the cartridge 22 and through the compartments 88 when the cartridge 22 is in use. However, it should be appreciated that in other embodiments the air intakes 134 may be arranged at different locations, such as through the cover 50, proximate the outlets 58, or any other reasonable location. In this manner, multiple dosages of the API may be contained and prepared for use by the user in the cartridge 22.

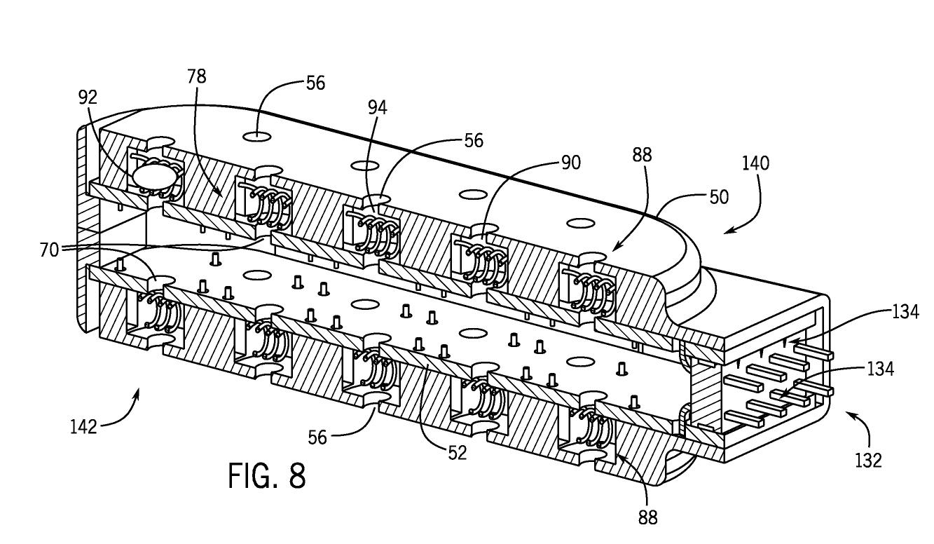

FIG. 8 is a cross-sectional view of the cartridge 22. In the illustrated embodiment, the connective end 132 is arranged to facilitate connection with the vaporizing device 10, for example, via pogo pins. Accordingly, the controller 28 can send information to the cartridge 22 and receive information from the cartridge 22. For example, the controller 28 may determine which compartments 88 have been used so that subsequent use of the cartridge 22 will utilize a different compartment 88.

As shown in FIG. 8, the compartments 88 are in a stacked configuration such that the respective outlets 58 are axially aligned along respective compartment axes 94. In other words, compartments 88 are arranged at a top 140 and a bottom 142 of the cartridge 22. However, it should be appreciated that the labeled top 140 and bottom 142 is not intended to limit the orientation of the cartridge 22 in operation. Each compartment 88 includes the chamber 90 for storing the wick 92 and also the heater 78 extending through the chamber 90. As described above, the heaters 78 are coupled to the respective bases 52 and receive electrical power from the power supply 26 via the connection through the connective end 132. In the illustrated embodiment, there are two bases 52 positioned within the cartridge 22. As described above, in certain embodiments, the base 52 is formed from a plastic material that is capable of withstanding high temperatures to thermally isolate adjacent compartments 88. Moreover, in certain embodiments, the base 52 is a printed circuit board (PCB). In this manner, individual compartments 88 may be activated (e.g., fired) and utilized without activating adjacent compartments 88. As used herein, activating the compartments 88 refers to vaporizing the API for inhalation. Moreover, in certain embodiments, activating the compartments 88 may be referred to as firing the compartments 88. However, firing the compartments should not be interpreted to mean igniting the compartments 88 such that a flame is generated within the compartments 88. Instead, firing is to be interpreted as activating. That is, firing the cartridge 88 refers to vaporizing the API in the cartridge.

As described above, the individual compartments 88 include the outlets 58 extending through the surface 56 of the cover 50 on both the top 140 and the bottom 142. Furthermore, as illustrated, the intakes 70 are arranged within the cartridge 22 along the base 52. It should be appreciated that, in certain embodiments, the air intakes 134 may be arranged through the cartridge 22 to enable air to flow through the interior of the cartridge to drive the API from the chamber 90 for inhalation by the user. However, in other embodiments, the activation and vaporization of the API may be sufficient to enable the usage by the user. For example, as described above, the API may be an oil that saturates the wick 92. As the oil is converted to gaseous form it will expand and flow out of the outlet 58 for subsequent inhalation by the user. As a result, additional air passages into the cartridge 22 may not be utilized.

In the illustrated embodiment, the top 140 and the bottom 142 of the cartridge 22 are in a spaced relationship to thereby separate the individual compartments 88 so that energy from the heater 78 of one compartment 88 is not transmitted to an adjacent or stacked compartment 88. Furthermore, the illustrated embodiment includes 10 compartments 88. However, as described above, the cartridge 22 may include any suitable number of compartments 88 to thereby hold metered dosage amounts of API.

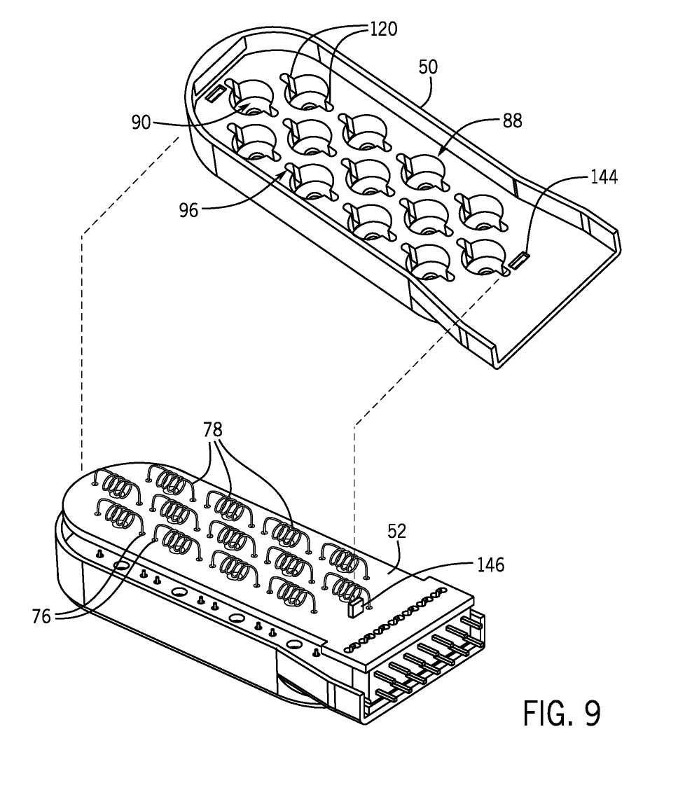

FIG. 9 is an exploded perspective view of the cartridge 22. In the illustrated embodiment, a top portion of the cover 50 is removed from the cartridge 22 to illustrate the position of the heaters 78 on the base 52. As shown, each compartment 88 includes an individual heater 78 that is individually activated to vaporize the API positioned within the chamber 90 of the compartment 88. In the illustrated embodiment, the heaters 78 extend through respective passages 76 to thereby receive activation energy from the base 52, for example, via the connective end 132 communicatively coupled to the power supply 26. Accordingly, the API may be individually vaporized in the compartments 88 for inhalation by a user.

In the illustrated embodiment, an interior portion of the cover 50 is illustrated. As described above, the compartments 88 include the chambers 90 and notches 120 for receive the heater 78. Moreover, the barrier 96 is at least partially built into the cover 50 to thermally isolate compartments 88 from adjacent compartments 88. Furthermore, in the illustrated embodiment, the cover 50 includes a slot 144 for receiving an EEPROM memory chip 146. As described above, in certain embodiments, information related to the cartridge 22 may be stored on the EEPROM memory chip 146, such as the type of API, the number of compartments 88, and the like. Furthermore, in certain embodiments, the EEPROM memory chip 146 may include programmable language to determine whether the cartridge 22 is a genuine cartridge specifically formulated for use with the vaporizing device. If the vaporizing device 10 determines the cartridge 22 is genuine, then the user may utilize the API. However, if the vaporizing device 10 determines the cartridge 22 is not genuine, the vaporizing device 10 may transmit a warning or error message to inform the user that usage of the cartridge 22 will not be performed with the vaporizing device 10. Accordingly, usage information for the cartridge 22 may be transmitted between the vaporizing device 10 and the cartridge, and in certain embodiments, to the user via a personal electronic device.

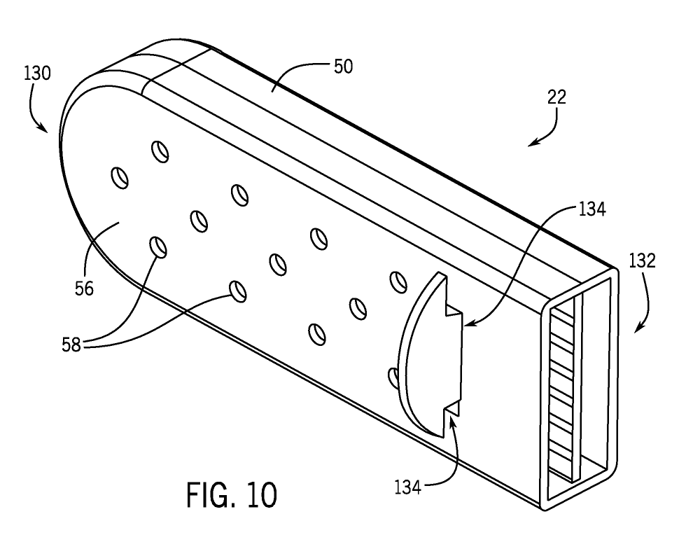

FIG. 10 is a perspective view of an embodiment of the cartridge 22. In the illustrated embodiment, the cartridge 22 includes the closed end 130 and the connective end 132. In certain embodiments, the connective end 132 facilitates coupling of the cartridge 22 to the vaporizing device 10. As described above, the outlets 58 are arranged along the surface 56 of the cover 50 of the cartridge. In certain embodiments, the outlets 58 are also arranged along the back side of the cartridge 22 (not visible in FIG. 10), thereby forming a stacked cartridge which will be described in detail below. As described above, the cartridge 22 includes multiple compartments 88 housing an individual, measured dose of API. In certain embodiments, each compartment 88 includes the individual heater 78 to facilitate vaporization of the API. Furthermore, as described above, in certain embodiments the cartridge 22 may receive an air intake to facilitate transportation of the vaporized API out of the cartridge 22. For example, as shown in FIG. 10, air intakes 134 may be along the cover 50. As a result, air may flow into the cartridge 22 and through the compartments 88 when the cartridge 22 is in use. However, it should be appreciated that in other embodiments the air intakes 134 may be arranged at different locations, such as proximate the connective end 132, proximate the outlets 58, or any other reasonable location. In this manner, multiple dosages of the API may be contained and prepared for use by the user in the cartridge 22.

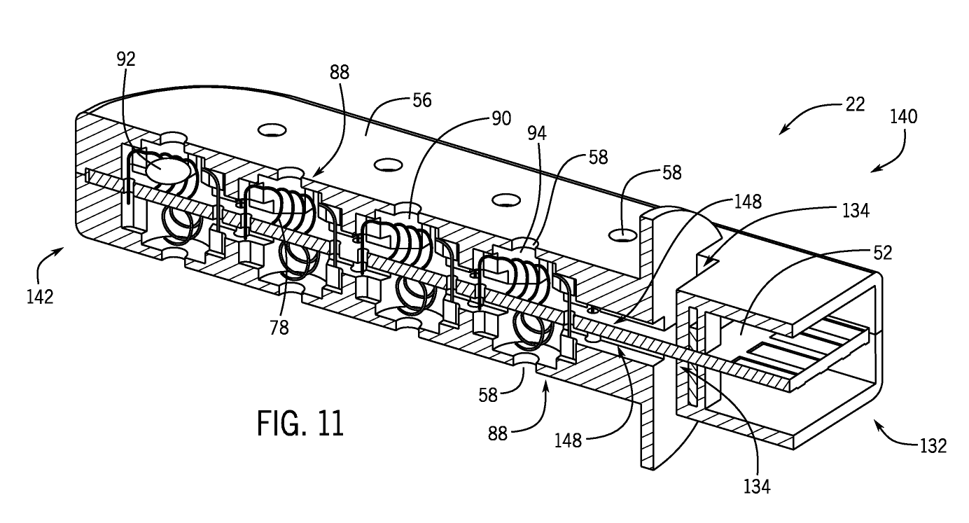

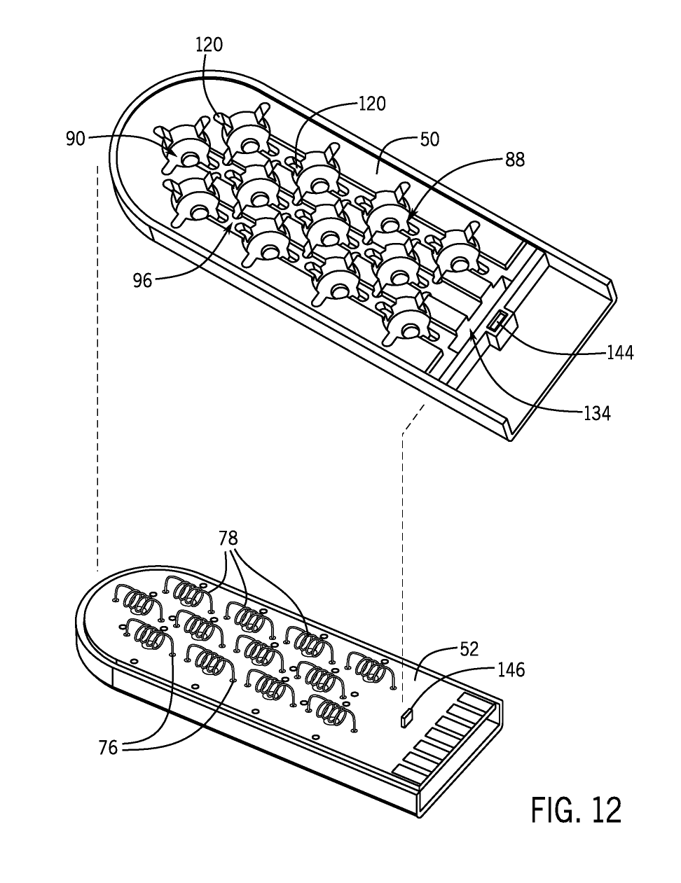

FIG. 11 is a cross-sectional view of the cartridge 22. In the illustrated embodiment, the connective end 132 is arranged to facilitate connection with the vaporizing device 10, for example, via pogo pins. Accordingly, the controller 28 can send information to the cartridge 22 and receive information from the cartridge 22. For example, the controller 28 may determine which compartments 88 have been used so that subsequent use of the cartridge 22 will utilize a different compartment 88.

As shown in FIG. 11, the compartments 88 are in a stacked configuration such that the respective outlets 58 are axially aligned along respective compartment axes 94. In other words, compartments 88 are arranged at a top 140 and a bottom 142 of the cartridge 22. However, it should be appreciated that the labeled top 140 and bottom 142 is not intended to limit the orientation of the cartridge 22 in operation. Each compartment 88 includes the chamber 90 for storing the wick 92 and also the heater 78 extending through the chamber 90. As described above, the heaters 78 are coupled to the base 52 and receive electrical power from the power supply 26 via the connection through the connective end 132. In the illustrated embodiment, there is one base 52 positioned within the cartridge 22. As described above, in certain embodiments, the base 52 is formed from a plastic material that is capable of withstanding high temperatures to thermally isolate adjacent compartments 88. Moreover, in certain embodiments, the base 52 is a PCB. In this manner, individual compartments 88 may be activated and utilized without activating adjacent compartments 88.

As described above, the individual compartments 88 include the outlets 58 extending through the surface 56 of the cover 50 on both the top 140 and the bottom 142. In the illustrated embodiment, the base 52 does not include the intakes 70, as shown above in FIG. 8. Instead, there is a flow path 148 arranged between the base 52 and the cover 50. That is, there is a gap between the base 52 and the chamber 90 to enable air entering through the air intakes 134 to drive the vaporized API out of the chamber 90 through the outlets 56. In the illustrated embodiment, the air intakes 134 extend through the cover 50 and are arranged to fluidly couple to the flow paths 148 on each side of the base 52. It should be appreciated that, in certain embodiments, the air intakes 134 and the flow paths 148 enable air to flow through the interior of the cartridge 22 to drive the API from the chamber 90 for inhalation by the user. However, in other embodiments, the activation and vaporization of the API may be sufficient to enable the usage by the user. For example, as described above, the API may be an oil that saturates the wick 92. As the oil is converted to gaseous form it will expand and flow out of the outlet 58 for subsequent inhalation by the user. As a result, additional air passages into the cartridge 22 may not be utilized.

In the illustrated embodiment, the top 140 and the bottom 142 of the cartridge 22 are in a spaced relationship to thereby separate the individual compartments 88 so that energy from the heater 78 of one compartment 88 is not transmitted to an adjacent or stacked compartment 88. For example, the base 52 may be utilized to thermally isolate the compartments 88. Additionally, the cover 50 may also be utilized to thermally isolate the compartments. Furthermore, the illustrated embodiment includes eight compartments 88. However, as described above, the cartridge 22 may include any suitable number of compartments 88 to thereby hold metered dosage amounts of API.

FIG. 12 is an exploded perspective view of the cartridge 22. In the illustrated embodiment, a top portion of the cover 50 is removed from the cartridge 22 to illustrate the position of the heaters 78 on the base 52. As shown, each compartment 88 includes an individual heater 78 that is individually activated to vaporize the API positioned within the chamber 90 of the compartment 88. In the illustrated embodiment, the heaters 78 extend through respective passages 76 to thereby receive activation energy from the base 52, for example, via the connective end 132 communicatively coupled to the power supply 26. Accordingly, the API may be individually vaporized in the compartments 88 for inhalation by a user.

In the illustrated embodiment, an interior portion of the cover 50 is illustrated. As described above, the compartments 88 include the chambers 90 and notches 120 for receive the heater 78. Moreover, the barrier 96 is at least partially built into the cover 50 to thermally isolate compartments 88 from adjacent compartments 88. In the illustrated embodiment, the barrier 96 may be formed as electrical insulation ribs. That is, the barrier 96 may not extend the depth of the chamber 90 to thereby form a portion of the flow path 148. Furthermore, in the illustrated embodiment, the cover 50 includes a slot 144 for receiving an EEPROM memory chip 146. As described above, in certain embodiments, information related to the cartridge 22 may be stored on the EEPROM memory chip 146, such as the type of API, the number of compartments 88, and the like. Furthermore, in certain embodiments, the EEPROM memory chip 146 may include programmable language to determine whether the cartridge 22 is a genuine cartridge specifically formulated for use with the vaporizing device. If the vaporizing device 10 determines the cartridge 22 is genuine, then the user may utilize the API. However, if the vaporizing device 10 determines the cartridge 22 is not genuine, the vaporizing device 10 may transmit a warning or error message to inform the user that usage of the cartridge 22 will not be performed with the vaporizing device 10. Accordingly, usage information for the cartridge 22 may be transmitted between the vaporizing device 10 and the cartridge, and in certain embodiments, to the user via a personal electronic device. Moreover, as illustrated, the air intake 134 extends through the cover 50 to thereby permit air flow through the interior of the cartridge 22.

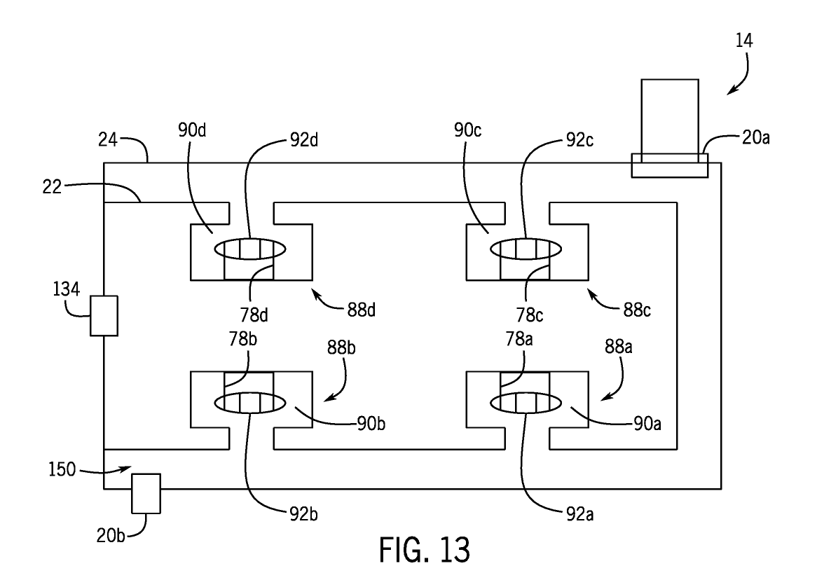

FIGS. 13-15 are schematic cross-sectional side views of the cartridge 22 arranged within the housing 24 during a usage cycle. It should be appreciated that the figures have been simplified for clarity and to focus the discussion of the usage cycle. FIG. 13 illustrates the cartridge 22 having the compartments 88 in the stacked configuration. That is, there are two compartments 88 on the top 140 and two compartments 88 on the bottom 142. However, as described above, it should be appreciated that, in other embodiments, there may be more or fewer compartments 88 and, moreover, the cartridge 22 may be generally flat and/or disc shaped as illustrated above. In the illustrated embodiment, the compartments 88a-88d include the wicks 92a-92d within the chambers 90a-90d. Furthermore, each compartment 88a-88d includes the respective heater 78a-78d for converting the API to an inhalable vapor. As illustrated in FIG. 13, the housing 24 substantially surrounds the cartridge 22 such that a void space 150 is positioned around the cartridge 22. However, in the illustrated embodiment, at least a portion of the cartridge 22 forms the void space 150. For example, as shown, the cartridge 22 may contact the housing 24 to facilitate formation of the void space 150. The void space 150 is positioned to receive and store the inhalable vapor from the respective chambers 90 after activation. In certain embodiments, the void space 150 creates an annulus about the cartridge 22 that substantially surrounds the cartridge 22 on each side. However, in other embodiments, the void space 150 may only be positioned on a certain side of the cartridge 22. It should be appreciated that the void space 150 is positioned to receive the vapor from the chambers 90a-90d and the general configuration and volume of the void space 150 may be particularly selected based on anticipated operating conditions. For example, the void space 150 may be sized to receive the vapor from one chamber 90, from two chambers 90, from three chambers 90, or any suitable number of chambers 90. In certain embodiments, the user may not be able to inhale all of the vapor produced from the respective chambers 90, and as a result, may need multiple puffs or pulls to administer the metered amount of API in each compartment 88. Accordingly, the flow restrictor 20a is arranged near the mouthpiece 14 to substantially block flow of the vapor to the mouthpiece 14 until one or more conditions are met. For example, as described above, the flow restrictor 20 may be a one-way valve that only opens when the user generates suction pressure at the mouthpiece 14. Moreover, the flow restrictor 20b is arranged on the housing 24 and enables air flow into the void space 150. For example, as the vapor produced by activating the compartment 88 fills the void space 150 and is subsequently inhaled through the mouthpiece 14, the pressure within the void space 150 is equalized via air entering the void space 150 through the flow restrictor 20b. Additionally, in certain embodiments, air flow may also enter the cartridge 22 through the air intake 134. It should be appreciated that, in certain embodiments, the air intake 134 may be communication with air outside of the housing 24 and/or with the void space 150.

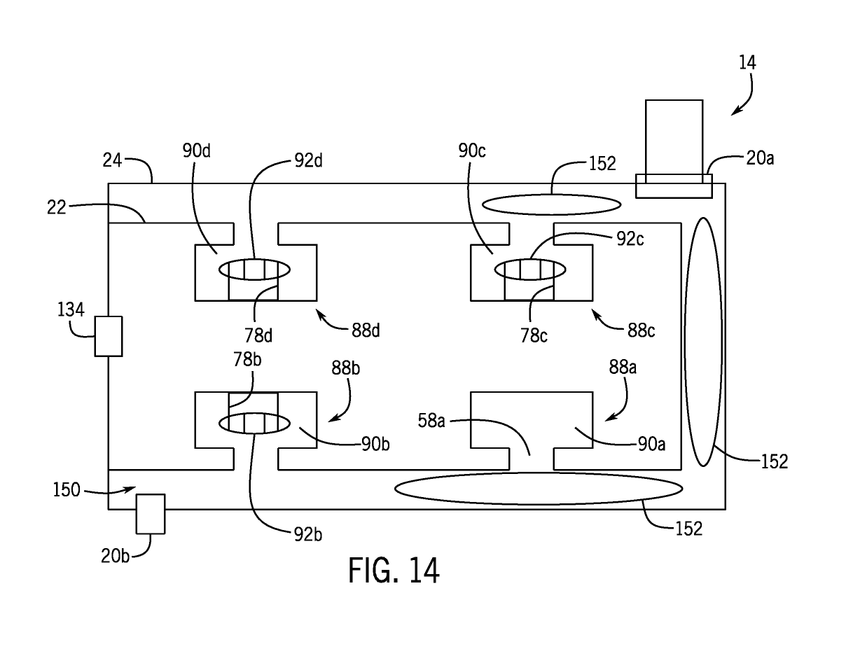

FIG. 14 illustrates the housing 24 filling with a vapor 152 after the heater 78a is activated. As described above, in operation, the power supply 26 transmits energy to the heater 78 to vaporize the API soaked into the wick 92. When the API is vaporized, the change in state from solid and/or solid/liquid expands the volume of the API and the vapor 152 flows out of the chamber 90a through the outlet 58a to thereby fill at least a portion of the housing 24. In the illustrated embodiment, the chambers 90b-90d still have their wick 92 and API because of the insulating properties of the cover 50. That is, individual compartments 88 may be activated without activating adjacent compartments 88. In the illustrated embodiment, the heater 78a is for a single use and burns up along with the wick 92. However, in other embodiments, the heater 78a may remain in the chamber 90 after activation. Furthermore, the wick 92 may remain after activation. That is, the compartment 88 may be heated such that the wick 92 is not burned or removed but the API is vaporized for inhalation.

In the illustrated embodiment, the vapor 152 is blocked in the void space 150 by the flow restrictor 20. That is, the vapor 152 is not free to flow out of the housing 24 until the flow restrictor 20a is positioned to enable flow to the mouth piece 14, for example, via suction pressure. As a result, the vapor 152 remains within the housing 24 until the user is ready to inhale the vapor 152. As described above, in certain embodiments, the user may not be able to inhale all of the vapor 152 at one time. For example, the user may have reduced lung capacity or prefer smaller puffs on the vaporizing device 10. Through placement of the flow restrictor 20a, the user may choose how much vapor 152 to inhale at a time and upon completion of the inhalation, the flow restrictor 20a can close and block the remaining portion of the vapor 152 from exiting the housing 24. In this manner, the user may determine how many puffs on the vaporizing device 10 to take to administer all of the metered API in the chambers 90.

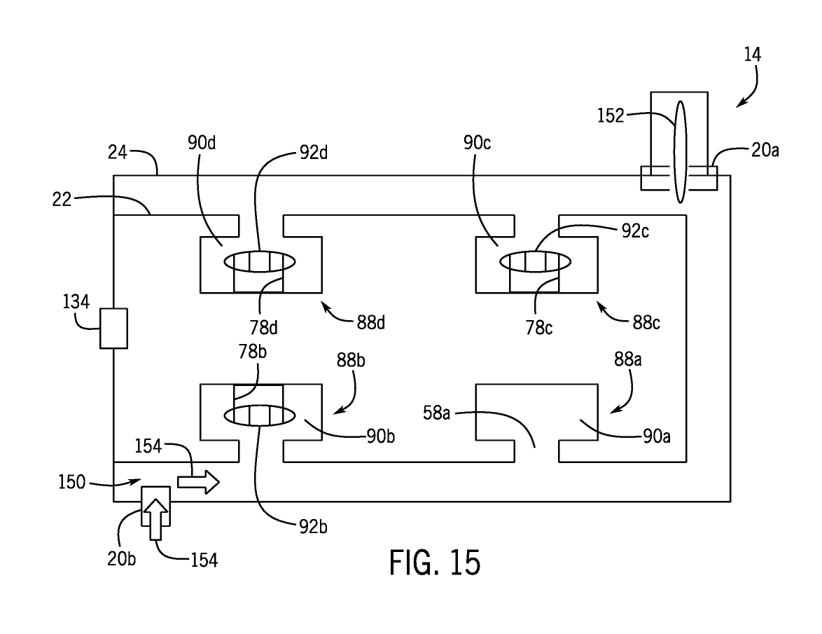

FIG. 15 illustrates the vapor 152 flowing through the open flow restrictor 20a through the mouthpiece 14. As described above, in certain embodiments, suction pressure at the mouthpiece 14, for example, via a user inhaling at the mouthpiece 14, may move the flow restrictor 20 to an open position to enable flow of the vapor 152 from the housing 24 and through the mouthpiece 14. In this manner, the user may inhale the vapor 152 for therapeutic use and, in certain embodiments, may prepare the vaporizing device 10 for activating a second chamber 90b-90d for additional administration of the API. In the illustrated embodiment, air flow represented by the arrows 154 equalizes the pressure in the void space 150 as the user inhales the vapor 152 via the mouthpiece 14.

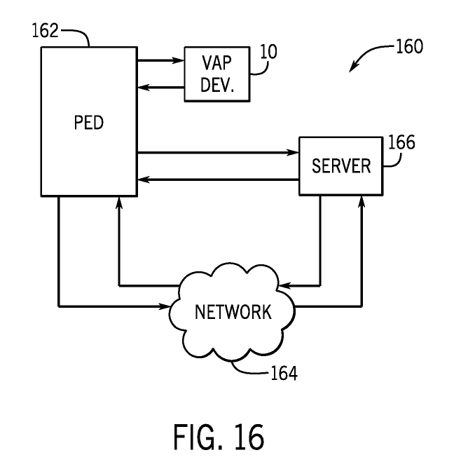

FIG. 16 is a schematic diagram of an embodiment of a communication network 160 coupling a personal electronic device (PED) 162, the vaporizing device 10, a network 164, and one or more servers 166. This communication network 160 enables tracking and evaluation of the use of the vaporizing device 10, such as dosage administration, tracking usage, tracking efficacy, storing medical information, making recommendations, communications with users/patients, and the like. Furthermore, in certain embodiments, the communication network 160 may be utilized to provide access to specific features for users, such as communities or blogs. For example, the communication network 160 may be utilized to direct users to a website or message board that includes information from a community of users. Additionally, users may be directed to blogs or postings that could include news, tips for use, advertisements for products, or other information. In the illustrated embodiment, the PED 162 is communicatively coupled to the vaporizing device 10, for example, via the communication device 134 of the vaporizing device 10. In certain embodiments, the PED 162 is a smart phone, wearable device, computer, or any other device suitable for facilitating communication between electronic devices. For example, the PED 162 may be communicatively coupled to the vaporizing device 10 via a BLUETOOTH transceiver; wireless internet transceiver, or near field communication transceiver. Moreover, in certain embodiments, the PED 162 may receive information regarding the composition of the API within the cartridge 22. As described above, the cartridge 22 may include the identification 110, such as an RFID tag, EEPROM, or a barcode, that may be evaluated and processed by the PED 162 and/or the vaporizing device 10. Accordingly, the PED 162 may receive and process the type of API being utilized by the user when a particular cartridge 22 is loaded into the vaporizing device 10.

In the illustrated embodiment, the PED 162 is communicatively coupled to the network 164. For example, the network 164 may be a cloud-based network that stores information for ready access by the PED 162 and/or the vaporizing device 10. Furthermore, in certain embodiments, the network 164 may be one or more servers that store information. For example, the network 164 may be a secure server that stores patient medical information, such as ailment and prescription information, for access by the PED 162 to determine prescribed dosing and administration procedures. In the illustrated embodiment, the network 164 is also communicatively coupled to the server 166. The server 166 may be utilized to store information and, in certain embodiments, directly communicates with the PED 162 and/or the vaporizing device 10. For example, the PED 162 may include a mobile application (e.g., an app) that tracks usage and efficacy of the API. The user may input information into the app, such as ratings or comments on the efficacy, which may be relayed to the server 166. Thereafter, the information on the server 166 may be utilized to recommend dosages or cartridges 22 to the user. For example, the user suffering from a particular ailment (e.g., depression, anxiety, chemotherapy related nausea, etc.) may load the app on the PED 162 and input their ailment. From there, the server 166 may evaluate information received from multiple users and evaluate the efficacy and usage statistics of a variety of cartridges 22. Furthermore, the server 166, via the app on the PED 162, may recommend one or more cartridges 22 and dosages to the user. For instance, a particular API may be used by a majority of individuals to treat anxiety. Accordingly, the server 166 may recommend the API to the user, thereby reducing the “guess and check” approach for the user finding the proper API to treat their respective ailments. Moreover, the server 166 may receive usage information from the PED 162 and send reminders to the user to either administer the dose or to remind the user to order additional cartridges 22. Furthermore, in certain embodiments the server 166 and/or the network 164 may be used to control use of the vaporizing device 10. For example, in certain embodiments the user may be prescribed a certain dosage over a period of time. Accordingly, once the user administers the dosage the vaporizing device 10 may transmit a signal to the PED 162, which may transmit a signal to the network 164 and/or the server 166 that the dosage has been administered. Accordingly, the vaporizing device 10 may be locked or prevented from administering another dosage until a predetermined period of time, for example, a number of hours or a particular time of day. For example, the network 164 and/or the server 166 may transmit a signal to the controller 28 of the vaporizing device 10 via the PED 162 that prevents use of the vaporizing device 10 until a certain period of time has passed, until a certain time of day, until another signal is received, or the like. In this manner, continued use of the vaporizing device 10 may be monitored and evaluated by the server 166 to streamline and record the process for users.

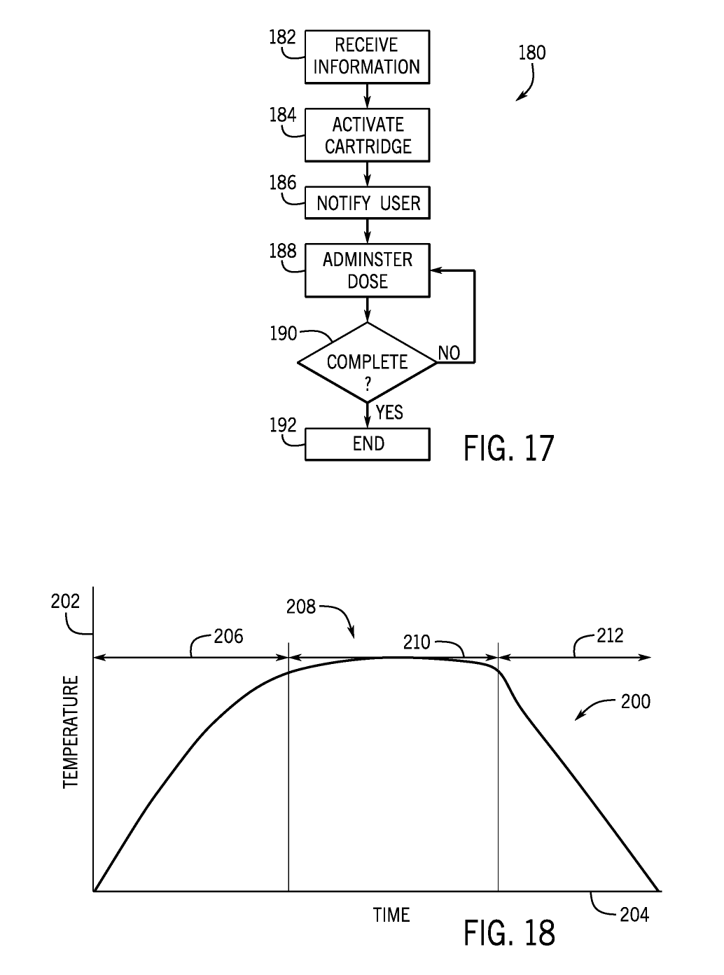

FIG. 17 is a flow chart of an embodiment of a method 180 for administering a dosage from the vaporizing device 10. For example, the user may install the cartridge 22 within the vaporizing device 10 and prepare to administer a dosage from the API arranged within the cartridge 22. As described above, in certain embodiments, the API saturates the wick 92 to provide a metered dosage amount in each individual compartment 88 of the cartridge 22. In the illustrated embodiment, cartridge information is received (block 182). For example, the user may scan the cartridge 22 with the PED 162 to determine the type of API positioned within the cartridge 22. Moreover, in certain embodiments, the cartridge 22 may include EEPROM memory with dosage and API information stored therein, which may be communicated to the vaporizing device 10 and transmitted to the PED 162, for example, via the controller 28, the communication device 34, and/or the display 40. Next, the compartment 88 is activated (block 184). For example, the user may press the switch 38, which transmits a signal to the power supply 26 to transmit energy to the cartridge 22. As described above, the controller 28 may determine which compartment 88 to transmit the energy to. That is, the controller 28 may track which compartments 88 have been activated and which compartments 88 still have API. As described above, the cartridge 22 may include multiple compartments 88 that are isolated from one another to thereby prevent transmission of heat between the compartments 88, and as a result, individual compartments 88 may be activated without activating adjacent compartments 88. As such, the API in the chamber 90 of the compartment 88 is heated.

Thereafter, the user is notified that the API is ready for inhalation (block 186). For example, a period of time may pass as the API is vaporized and the vapor 152 fills the void space 150. Because each compartment 88 includes the metered dosage, the vaporizing device 10 may transmit a signal (e.g., a sound, a vibration, a flashing light, etc.) to notify the user when vaporization is complete and the metered dosage is substantially fully vaporized. For example, the auxiliary component 36 may include a vibration device that vibrates to notify the user that vaporization is complete. Moreover, the display 40 may show a message or flash colors (e.g., red during vaporization and green when complete) to notify the user.

Thereafter, the dosage is administered (block 188). For example, the user may inhale the vapor 152 produced from the chamber 90 via the mouthpiece 14. As described above, in certain embodiments, the vaporizing device 10 includes the flow restrictor 20 to block flow of the vapor 152 to the mouthpiece 14 until a certain condition is met, such as suction pressure at the mouthpiece 14. In this manner, the user may administer the dosage at a rate that is convenient to the user. For example, the user may inhale all of the vapor 152 at one time. However, in other embodiments, the user may take multiple puffs on the vaporizing device 10 to inhale the vapor. Next, the presence of vapor 152 is evaluated to determine if the dosage is complete (operator 190). For example, the user may look at the housing 24 to determine if additional vapor 152 is present. Moreover, the user may attempt an additional inhalation to determine whether the vapor 152 is present. Furthermore, the one or more sensors of the auxiliary component 36 may detect the presence of vapor 152, for example, via a photoelectric (e.g., infrared, visible, ultraviolet, etc.) or ionization detector. Thereafter, the display 40 may notify the user whether additional vapor 152 is present. If additional vapor is present, then the dosage is administered (block 188). If additional vapor is not present, the process ends (block 192). For example, the user may turn off the vaporizing device 10. Moreover, the user may input information into the app on the PED 162 to indicate the dosage was administered and/or to report on the efficacy of the dosage. Accordingly, the user can monitor and administer API from the vaporizing device 10.

FIG. 18 is a graphical representation of the API being heated to enable vaporization of the API. As described above, in certain embodiments, the API is in the form of an oil, which may be viscous, that saturates the wick 92 and is positioned within the chamber 90 prior to activation and vaporization of the API. In certain embodiments, the oil may harden during shipment and storage, and therefore be more difficult to vaporize. If high heat is applied directly, the API may be scorched or burn inefficiently, leading to a poor taste or loss of API. However, if the API is gradually heated to reduce the viscosity, and then heated to enable vaporization, the integrity of the API may be maintained. FIG. 18 illustrates a curve 200 illustrating the heating of the API over time. The vertical axis 202 represents temperature and the horizontal axis 204 represents time. In a first period of time 206, the API is gradually heated. Then, once the API has been heated to approximately a desired temperature 208, for example, a temperature that reduces the viscosity, the API is held at that temperature (e.g., the desired temperature 208) for a second period of time 210. The API is vaporized over the second period of time 210 to enable inhalation by the user. Then, the temperature within the chamber 90 is decreased over a third period of time 212. For example, the power supply 26 may cease providing energy to the heater 78, thereby enabling the temperature to reduce over the third period of time 212. In this manner, the API may be efficiently heated for use by the user.

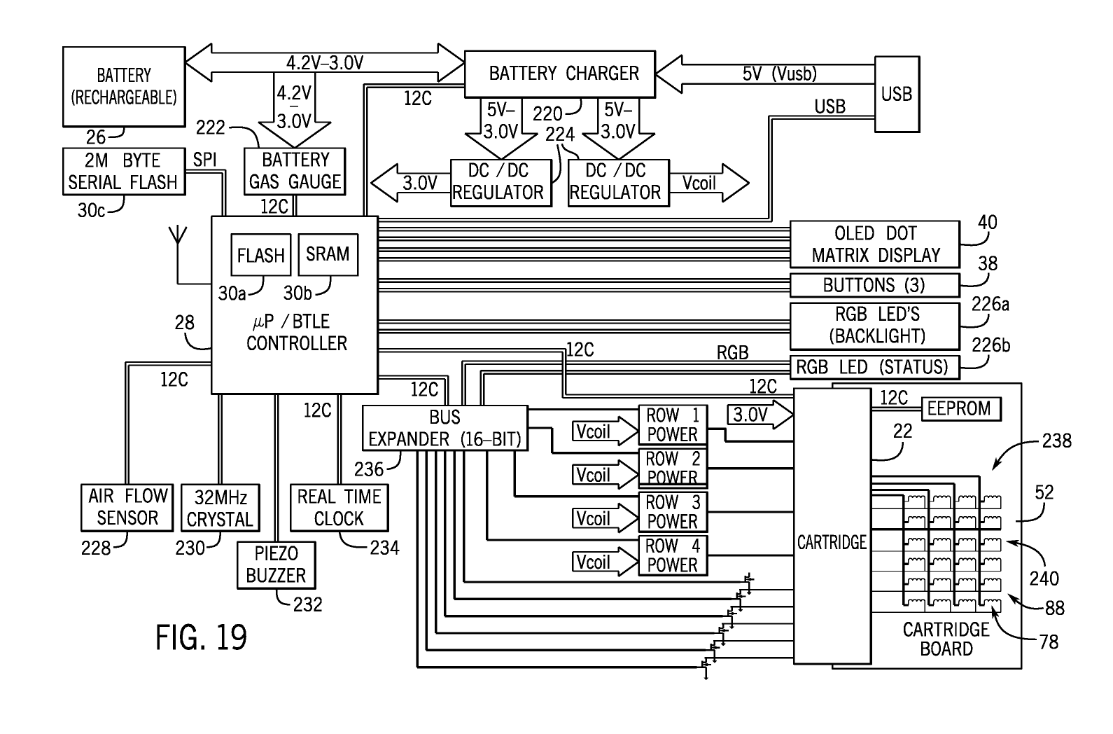

FIG. 19 is a schematic diagram of an embodiment of hardware architecture for the vaporizing device 10. As described above, in certain embodiments, the vaporizing device 10 includes the power supply 26, illustrated as a battery in FIG. 19. In the illustrated embodiment, the power supply 26 is communicatively coupled to a charger 220. For example, the charger 220 may transmit electrical energy to the power supply 26, for example, via an outlet connection or a universal serial bus connection. Accordingly, electrical energy may be transmitted to the power supply 26 for use with vaporizing the API in the cartridge 22 or for performing various operations with the vaporizing device 10, such as monitoring battery levels, notifying the user when API is vaporized, or the like. In the illustrated embodiment, the power supply 26 is further communicatively coupled to a gas gauge 222. That is, in embodiments where the power supply 26 is a battery, the gas gauge 222 may monitor operation of the battery and facilitate venting and/or temperature control to enhance operations and/or improve longevity. Furthermore, in the illustrated embodiments, regulators 224 are coupled to the charger 220. In the illustrated embodiment, the regulators 224 are DC/DC regulators, however, it should be appreciated that other regulators may be used.

As described in detail above, the vaporizing device 10 includes the controller 28. In the illustrated embodiment, the controller 28 includes onboard Flash and SRAM memory 30a, 30b. Furthermore, the controller 28 is communicatively coupled Serial Flash memory 30c. As such, the controller 28 may store executable instructions on the various memories 30 to thereby facilitate operation and control of the vaporizing device. Furthermore, as described above, the controller 28 is communicatively coupled to the display 40, switches 38, and indicators 226. For example, in the illustrated embodiment, the display 40 is an OLED dot matrix display. Furthermore, the switches 38 includes multiple buttons, for example, three buttons in the illustrated embodiment. Additionally, the indicators 226a, 226b include LEDs that may provide indications related to the status of the vaporizing device 10 (e.g., whether the API is vaporized and ready for use) or the like.

In certain embodiments, the vaporizing device 10 includes the auxiliary components 36. For example, an air flow sensor 228 may be arranged within the vaporizing device 10 to determine when the user is inhaling API at the mouthpiece 14. As shown, the air flow sensor 228 is communicatively coupled to the controller 28 to enable for the transmission of information. For example, upon activation of the air flow sensor 228, the controller 28 may transmit a signal to activate the second flow restrictor 20b (FIG. 15) to enable air to flow into the vaporizing device 10 to equalize pressure due to the inhalation of the vapor 152. Additionally, auxiliary components 36 may include a 32 MHz crystal 230, a piezo buzzer 232, and a clock 234. For example, the piezo buzzer 232 may transmit an auditory alarm to indicate the vaporizing device 10 is ready to use. Furthermore, the clock 234 may be utilized to track dosage times and/or as an alarm to remind the user to take their dosage. In this manner, functionality of the vaporizing device 10 may be improved to enhance the user experience.

Additionally, as described above, in certain embodiments the cartridge 22 is communicatively coupled to the controller 28. For instance, as shown in FIG. 19, the cartridge 22 includes the base 52 formed from a printed circuit board that is coupled to the controller 28 via a bus expander 236. As described above, the cartridge 22 includes the compartments 88 which house the API for vaporization and use. In the illustrated embodiment, the compartments 88 are arranged in a matrix having rows 238 and columns 240. As illustrated, there are 24 compartments 88 arranged in a 4×6 matrix. As will be described below, the controller 28 may transmit signals to the cartridge 22 to fire and/or activate certain compartments 88 and track the remaining compartments 88. In the illustrated embodiment, the cartridge 22 includes the identification 110 in the form of EEPROM. As such, information about the cartridge 22, such as the type of API, the metered dosage, creation date, and the like may be stored and transmitted to the controller 28.

In the illustrated embodiment, the compartments 88 include individual heaters 78 (illustrated as coils) that are coupled to the controller 28 and that receive electrical energy to convert to heat energy to vaporize the API stored in the wicks 92 (not pictured). In operation, the controller 28 will receive a signal, for example, from the switch 38, to activate a cartridge 88. From there, the controller 28 will transmit a signal to send electrical energy to a compartment, for example, the compartment at location 1×1 of the 4×6 matrix illustrated in FIG. 19 via the regulator 224. The heater 78 will vaporize the API, thereby enabling the user to inhale the vapor 152 via the mouthpiece 14 (for example, as illustrated in FIGS. 13-15). Thereafter, the controller 28 may store information to indicate which compartments 88 have been used. For example, the controller 28 may store information indicating that that the compartment 88 at the 1×1 location is used, and upon the next signal to activate a second compartment 88 may transmit the signal to the compartment 88 at the 1×2 location. Accordingly, the controller 28 may store and control activation of the compartments 88.

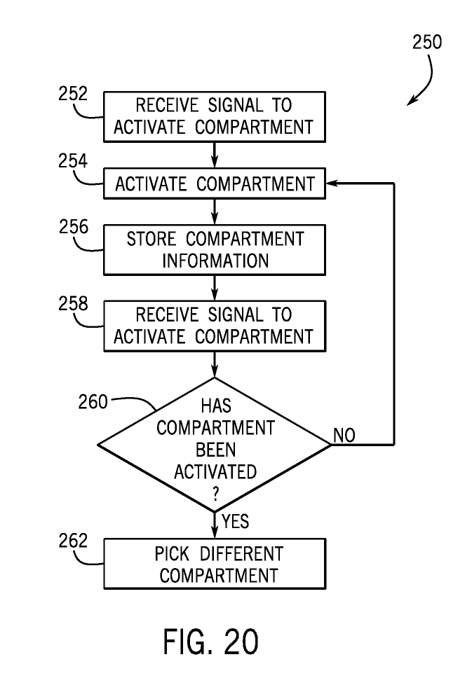

FIG. 20 is a flow chart of a method 250 for managing firing and use of the cartridge 22. For example, as described above, in certain embodiments, the cartridge 22 may include a matrix of compartments 88 arranged in a R×C matrix, where R is the number of rows and C is the number of columns (for example, the 4×6 matrix illustrated in FIG. 19). Upon receiving a signal to activate the compartment 88 (block 252), for example, via the switch 28, the cartridge 22 transmits a signal to transfer energy to a respective compartment 88 to active the API (block 254). For example, the controller 28 may transmit a signal to a respective heater 78 within the compartment 88. In certain embodiments, the cartridge 22 may begin at a first compartment 88, represented by 1×1 in the 4×6 matrix illustrated in FIG. 19. Thereafter, the controller 28 stores the compartment 88 information (block 256). For example, the controller 28 may store information indicative that compartment 88 corresponding to position 1×1 in the 4×6 matrix illustrated in FIG. 19 has been activated, and therefore will not transmit additional electrical energy to the compartment 88. Next, the controller 28 receives a second signal containing instructions to activate a respective compartment 88 (block 258). Upon receipt of the second signal, the controller 28 determines which compartments 88 have been activated and which have not. As a result, the controller 28 checks if a compartment 88 before transmitting a second to activate the respective compartments 88 (operator 260). If the compartment 88 has not been activated, the method 250 returns to block 254 to activate the compartment 88. However, if the compartment 88 has been activated, then a different compartment 88 is chosen (block 262). For example, the 1×2 compartment of the matrix illustrated in FIG. 19. Accordingly, the controller 28 may continuously cycle and move through the matrix of compartments 88 in order to prepare activation of the respective compartments 88. In certain embodiments, the controller 28 may cycle through the matrix based on columns. For example, the controller 28 may include programmed logic to cycle through the compartments 88 of the cartridges 22. That is, the programmed logic may add one to each column as the compartments 88 are activated. For example, for the 4×6 matrix of FIG. 19, the activation order may be 1×1; 1×2; 1×3; 1×4; 1×5; 1×6; 2×1; 2×2; and so on. In this manner, the compartments 88 may be efficiently tracked. However, in certain embodiments, the compartments 88 may be cycled through by row. That is, for the 4×6 matrix of FIG. 19, the activation order may be 1×1; 2×1; 3×1; 4×1; 2×1; and so on. Accordingly, the cartridge 22 may be monitored and fired in a particular order to ensure proper heat distribution and also to track remaining dosages for users.

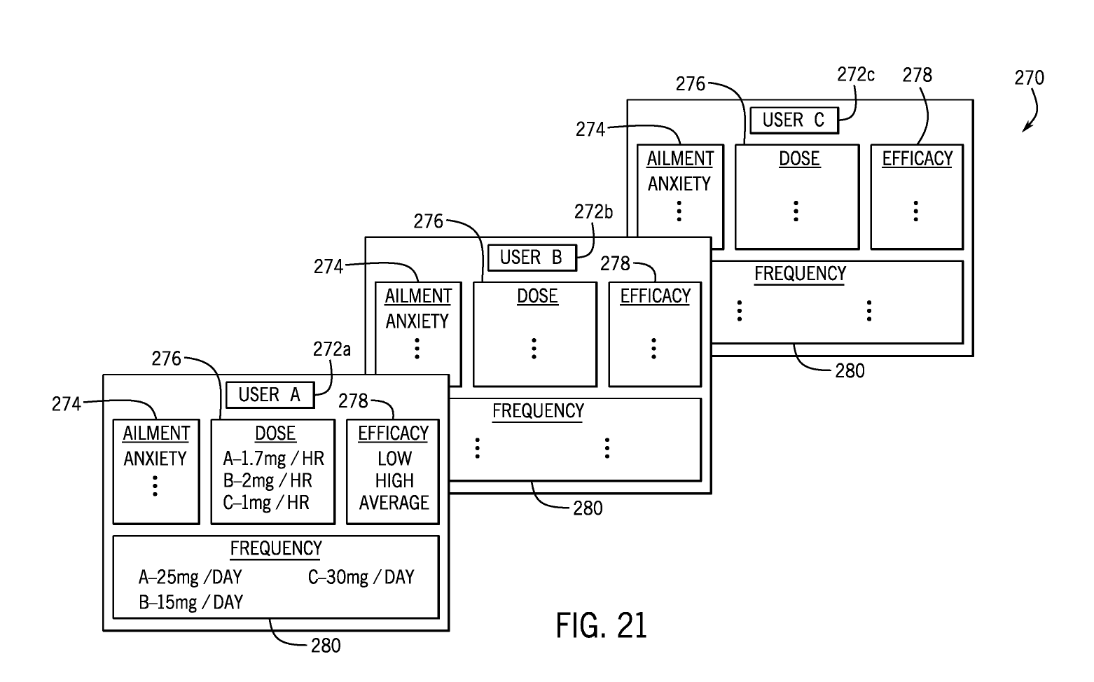

FIG. 21 is a schematic diagram of an embodiment of user profiles 270 generated by an authorized user of the vaporizing device 10. In certain embodiments, the user profile 270 will be generated through a computerized application that is executable on the personal electronic device 162. For example, upon receiving a prescription to utilize medicinal cannabis from a licensed medical professional, a user 272 (e.g., user 272a, user 272b, user 272c) may receive a passcode to enable the download and installation of the application. Thereafter, the user 272 may establish the user profile 270 to list ailments 274 to be treated by the medicinal cannabis and/or other API. It should be noted that, while the illustrated embodiment includes treatment utilizing medicinal cannabis, in other embodiments the API may be tobacco, an anti-inflammatory, a vaccine, or any other suitable API that can be vaporized and/or inhaled by the user. By way of example only, in the illustrated embodiment, anxiety is listed as an ailment for the user 272a. However, any other number of ailments may be associated with the user profile 270. Moreover, in the illustrated embodiment, each ailment 274 has an associated oil/dose identifier 276. That is, the oil/dose identifier 276 lists the API and the user dosages utilized by the user 272 for the given ailment 274. In certain embodiments, the user dosage is the unique dosage each user determines provides the proper efficacy to treat their unique ailment. The user dosage is determined via analysis of the efficacy of different APIs and adjustments (e.g., increasing or decreasing the dosage) based on the user's feedback. For example, the user 272 may utilize the API on the wick 92 in a compartment 88 and feel relief from an ailment for a period of time. However, the period of time the user 272 feels relief may be shorter than anticipated. For example, the API may recommend a certain dosage each hour. The user's experience, however, may differ and proper efficacy of the dosage may be at a certain dosage each half hour. Accordingly, the user profile 270 may be updated to reflect the user's individual dosage requirements after use and communication through the application. Moreover, the illustrated user profile 270 includes efficacy 278 for the given API and dosages identified in the dose identifier 276. In this manner, the user 142 may quickly and efficiently identify the API and user dosages that have been utilized to treat the given ailment 274. Moreover, the user 272 can identify the efficacy of the API and user dosages for further diagnosis and refinement by their medical practitioner.

Furthermore, in the illustrated embodiment, a frequency 280 is also included within the user profile 270. The frequency 280 is correlated to the total amount of API (e.g., cannabis, tobacco, anti-inflammatory, etc.) utilized by the user 272 over a period of time. For example, the frequency 280 may measure dosages per day, per week, per month, or any other suitable time frame. In this manner, the user's treatment plan can be continuously monitored and updated by their medical professional. Additionally, the frequency 280 may track each administered dosage of the vaporizing device 10 for the user. For example, in certain embodiments, use of the vaporizing device 10 may be unrestricted, thereby allowing the user to administer dosages as often as deemed necessary to treat one or more ailments. The vaporizing device 10 may record and transmit each dosage to the server 166 and/or personal electronic device 162. In certain embodiments, each dosage event includes a date and time stamp. The user profile 270 may save these dosage events to track and evaluate use of the vaporizing device 10 by the user. In this manner, the data collected may be utilized to improve the treatment plan for the user. Furthermore, transmission of the frequency 280 data (e.g., via the communication device 34) may be utilized to remind the user 272 to place an order for more cartridges 22. For example, if data indicates that the user 272 purchased the cartridge 22 with approximately 200 mg of API, and the frequency 280 indicates that the user 272 uses approximately 25 mg per week, it can be extrapolated that the cartridge 22 will last approximately eight weeks. Therefore, a notification may be sent to the user 272 (e.g., via the computerized application) to place an order when the supply is running low. Accordingly, the interruption of treatment (e.g., via running out of API) may be reduced, thereby providing improved care to the users 272.

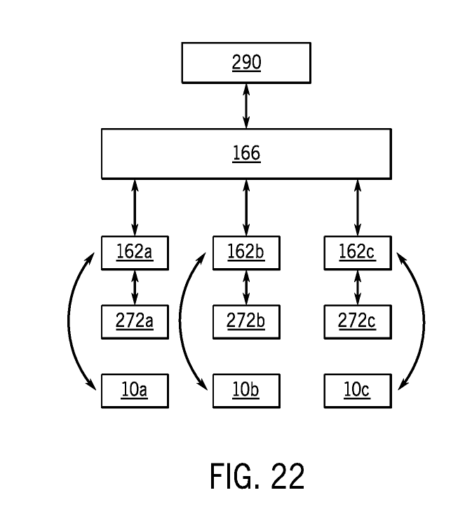

FIG. 22 is a schematic diagram of an embodiment of the server 166 that communicates with one or more personal electronic devices 162. It should be appreciated that the server 166 includes one or more memories and processors capable of utilizing machine-readable code to perform one or more computerized functions. As described above, the server 166 may contain the machine-readable code that includes written instructions to execute a computer application on the personal electronic device 162. Upon receiving authorization to download the computer application (e.g., a prescription to utilize medicinal cannabis, purchasing the vaporizing device 10, etc.), the user 272 sends a signal to the server 166 via the personal electronic device 162 to receive the computer application. Thereafter, the user 272 can interact with the server 166 via the personal electronic device 162 to send and receive information related to administering a dosage via the vaporizing device 10. For example, the user 272 may answer one or more questions regarding the efficacy of the dosage. Furthermore, the user 272 may enter information regarding their ailments 274, biometric data (e.g., height, weight, body mass index, etc.), pair one or more wearable fitness devices, or any other information that may be utilized to tailor the dosage to provide relief for the one or more ailments 274. As described above, the quantity of API within each compartment 88 of the cartridge 28 may be fixed, but the dosage may also be used to refer to the number of dosages (e.g., compartments 88 fired) during a period of time.

Moreover, as illustrated in FIG. 22, the vaporizing device 10 can communicate with the personal electronic device 162 via the communication device 34. For example, in certain embodiments, the personal electronic device 162 may transmit information related to the time of day the user 272 administered the dosage, the number of dosages administered by the user 272 over a period of time (e.g., a day, a week, a month), the energy remaining in the power supply 26, or any other reasonable information. As a result, in certain embodiments, the personal electronic device 162 may communicate directly with the server 166 to transmit information related to the administration of the dosage.

As shown, the server 166 is positioned to receive information from one or more users 272a, 272b, 272c via respective personal electronic devices 162a, 162b, 162c and/or from one or more vaporizing devices 10a, 10b, 10c. Furthermore, the server 166 is also communicatively coupled to a controller 290 that has access to modify one or more properties of the server 166. For example, the controller 290 may be a computer arranged to evaluate the feedback received from the users 272 and/or vaporizing devices 10 to update dosage profiles for particular APIs and/or provide unique user dosages to the users 272 based on their feedback. However, as described above, in certain embodiments the personal electronic device 162 may include the information (e.g., via downloading database) to evaluate and provide the unique user dosages. As a result of having access to the server 166, the controller 290 may evaluate feedback from multiple users each having one or more of the same ailments and each using one or more of the same API to treat the ailments. By processing the efficacy of certain APIs against certain ailments over a number of users over a period of time, the controller 290 may continuously update the dosage profiles to provide relief for the one or more ailments. That is, a recommended dosage may be adjusted based on feedback from multiple users over a period of time to enhance administration of the dosage for future users.

As described above, in certain embodiments the vaporizing device 10 transmits information to the personal electronic device 162 and/or to the server 166. For example, when in use, the processor 32 of the vaporizing device 10 may record at least one dosing property, such time of day of dosing or the like. Thereafter, the communication device 34 may transmit the at least one dosing property to the personal electronic device 162 for processing, evaluation, or record keeping purposes. For example, the dosing property may be the time of day of inhalation. Therefore, the personal electronic device 162 may transmit a signal to the vaporizing device 10 to “lock” or prevent use of the vaporizing device 10 until a certain interval of time has passed. However, in certain embodiments, the vaporizing device 10 may enable unrestricted use. That is, the user may administer as many dosages as the user deems necessary while the vaporizing device 10 monitors and tracks information related to the dosages, such as frequency, time of date, and the like.

Furthermore, as described above, the server 166 may be utilized to evaluate the feedback and output unique user dosages to the users 272. For example, upon receiving the feedback from the users 272, the server 166 and/or the controller 290 may analyze the data (e.g., via the one or more processors and memories) to determine whether or not to increase or decrease the dosage. After evaluating the data, the recommended user dosage may be transmitted to the user 272 and the user profile 270 may be updated for certain APIs. To this end, the user dosage may be continuously updated to provide adequate care for the ailments 144 of the user 272. Furthermore, as described above, in certain embodiments the personal electronic device 162 may evaluate the feedback and update the user profile 270 based on the feedback.

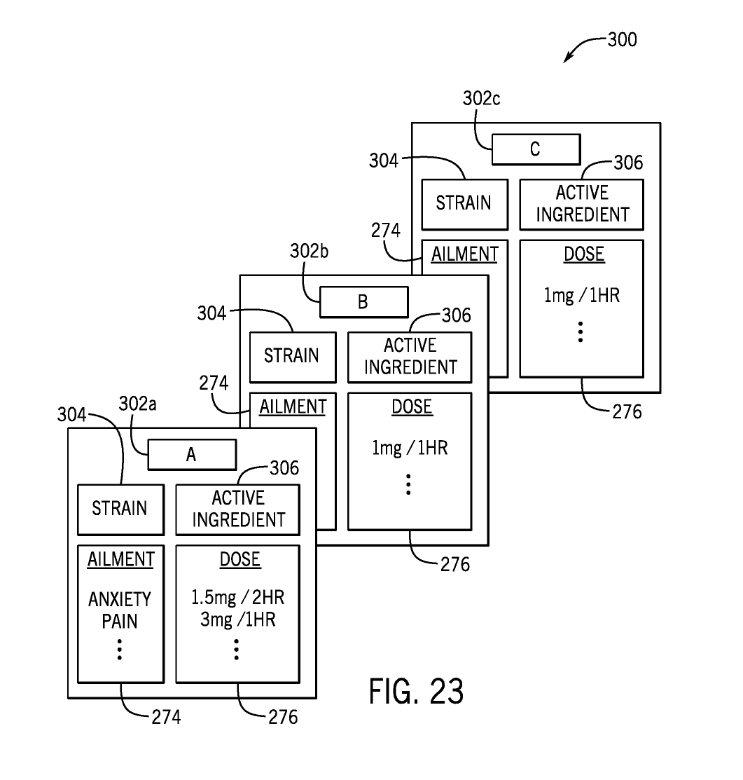

FIG. 23 is a schematic diagram of an embodiment of dosage profiles 300 generated for APIs 302a, 302b, 302c. As shown, each dosage profile 300 may include the strain 304 (e.g., cannabis, tobacco, etc.) utilized to formulate the API 302 and the quantity of active ingredient 306 in each cartridge 22 and/or compartment 88. Moreover, it should be appreciated that, in certain embodiments, the strain 304 can be correlated to the API, such as an anti-inflammatory or vaccine. For example, as described above, in certain embodiments the API 302 is formulated to contain approximately 200 mg of active ingredient (e.g., active THC, nicotine, anti-inflammatory, etc.) in each cartridge 22. This may be further broken down to identify the quantity of API 302 in each compartment 88 of the cartridge 22. For example, in embodiments where the cartridge 22 includes 25 individual compartments 88 and 200 mg of active ingredient, each compartment would contain 4 mg of active ingredient. Moreover, in the illustrated embodiment, the dosage profile 300 includes treated ailments 274 and recommended dosages 276. As described above, because the quantity of API is fixed within the compartments 88, the recommended dosage may be excluded or correlated to the number of compartments 88 fired in a given period of time. By way of example only, in the illustrated embodiment for API 302a, two of the treated ailments include anxiety and pain. Moreover, also by example, the associated recommended dosages are 1.5 mg every 2 hours and 3 mg every hour, respectively. For example, if each compartment 88 included 1.5 mg of active ingredient, the user would fire one cartridge every 2 hours to obtain the 1.5 mg or 2 cartridges every hour to obtain 3 mg. Accordingly, each API 302 may have a corresponding dosage profile 300 to effectively categorize and organize ailments 274 and recommended dosages.

As described in detail above, the vaporizing device 10 includes the cartridge 22 arranged within the housing 24. In certain embodiments, the cartridge 22 includes individual compartments 88 containing a metered amount of API, for example, saturated on the wick 92, and the heater 78. During operation, the heater 78 transmits energy to the wick 92, thereby vaporizing the API for inhalation by the user. In certain embodiments, the vapor 152 flows out of the chamber 90 of the compartment 88 and into the housing 24. The housing 24 holds the vapor 152 until the user is prepared for inhalation. For example, the housing 24 may be fluidly coupled to the mouthpiece 14 with the flow restrictor 20 blocking flow to the mouthpiece 14 until a certain condition is met, such as a suction pressure at the mouthpiece 14. In this manner, the user can control the amount of API inhaled through activation of one or more compartments 88, as well as how many inhalations or puffs the user uses to inhale the vapor 152. Furthermore, as described above, in certain embodiments, the vaporizing device 10 includes the controller 28 for sending and/or receiving information or instructions. For example, the controller 28 may monitor which compartments 88 are activated and relay the total number remaining to the user via the display 40. Moreover, the controller 28 may further be utilized to receive instructions from the user and/or the PED 162.

The foregoing disclosure and description of the invention is illustrative and explanatory of the embodiments of the invention. Various changes in the details of the illustrated embodiments can be made within the scope of the appended claims without departing from the true spirit of the invention. The embodiments of the present invention should only be limited by the following claims and their legal equivalents.

1. An apparatus, comprising:
a base;
a cover arranged over the base, the cover comprising a plurality of compartments for storing an active pharmaceutical ingredient (API) within a chamber of each compartment of the plurality of compartments, each chamber being formed within the cover;
a plurality of heaters, each respective heater of the plurality of heaters being positioned within the compartment for transmitting energy to the API; and
power contacts positioned on the base, the power contacts positioned to transmit received operational power to the plurality of heaters.

2. The apparatus of claim 1, comprising a plurality of outlets extending through the cover, wherein an outlet of the plurality of outlets is aligned with a respective chamber of the plurality of compartments such that API exits the respective chamber through the respective outlet when the API is converted to a vapor by the respective heater.

3. The apparatus of claim 1, comprising a connective end electrically coupled to the power contacts, the connective end having one or more leads to facilitate connection and communication with a power source.

4. The apparatus of claim 1, comprising a plurality of intakes, wherein an intake of the plurality of intakes extends through the base and is aligned with a respective chamber of the plurality of chambers, the intake directing an air flow through the chamber to drive the API out of the chamber via an outlet extending through the cover.

5. The apparatus of claim 1, comprising a barrier arranged between adjacent compartments of the plurality of compartments, the barrier formed from a heat resistant material to substantially block heat transfer between adjacent compartments.

6. The apparatus of claim 5, wherein the barrier forms at least a portion of the cover.

7. The apparatus of claim 1, wherein the base comprises a plurality of passages extending therethrough and each heater of the plurality of heaters extends through the passages.

8. A system for vaporizing an active pharmaceutical ingredient (API), comprising:
a body, the body comprising:
a housing positioned within the body, the housing arranged to receive vaporized activate pharmaceutical ingredients (APIs) for inhalation;
a mouthpiece arranged at a first end of the body, opposite a second end, the mouthpiece being fluidly coupled to the housing to direct the vaporized API out of the housing; and
a power supply to facilitate vaporization of the API; and
a cartridge for storing the API, the cartridge being positioned within the housing and comprising:
a base having a plurality of power contacts to electrically couple the cartridge to the power supply,
a cover arranged over the base, the cover comprising a plurality of compartments for storing the API, each compartment having a chamber; and
a plurality of heaters, wherein a heater of the plurality of heaters is positioned within a compartment of the plurality of compartments for transmitting energy from the power supply to the API in the chamber.

9. The system of claim 8, wherein each compartment of the plurality of compartments comprises an outlet extending through the cover, the outlet being fluidly coupled to the chamber to direct vaporized API out of the chamber and into the housing.

10. The system of claim 8, wherein the cartridge further comprises an identification, the identification providing information related to the type of API stored within the compartments.

11. The system of claim 8, further comprising a switch arranged on the body, the switch being communicatively coupled to the power supply to direct the power supply to transmit electrical energy to one or more heaters of the plurality of heaters to vaporize the API stored in the respective chambers.

12. The system of claim 8, wherein the plurality of compartments are arranged in a stacked configuration, the base being positioned between stacked compartments when the compartments are in the stacked configuration.

13. The system of claim 8, further comprising a flow restrictor, the flow restrictor positioned between the housing and the mouthpiece to regulate flow between the housing and the mouthpiece.

14. The system of claim 13, wherein the flow restrictor is a one way valve that moves from a closed position blocking flow to the mouthpiece to an open position enabling flow to the mouthpiece upon detection of a suction pressure at the mouthpiece.

15. The system of claim 8, further comprising a processor positioned within the body, the processor configured to monitor usage of the plurality of compartments of the cartridge and record when a compartment of the plurality of compartments is activated to vaporize the API.

16. The system of claim 15, wherein the processor transmits a signal to activate a compartment of the plurality of compartments that has not been activated and relays activation information to a user.

17. The system of claim 8, further comprising a communication device to transmit at least one piece of dosing information to at least one portable electronic device.

18. A method for using a vaporizing device, comprising:
receiving information from an identification arranged on a cartridge containing an active pharmaceutical ingredient (API);
activating a first compartment of a plurality of compartments arranged within the cartridge that house the API, wherein activating comprises converting the API into an inhalable vapor;
administering the dosage for inhalation by a user; and
preparing a second compartment, different from the first compartment, for a second activation.

19. The method of claim 18, further comprising storing the inhalable vapor in a housing before administering the dosage, the housing surrounding the cartridge.

20. The method of claim 18, further comprising determining whether the dosage has been fully administered by inspecting the housing for inhalable vapor after the dosage is administered.
